(19) United States
(12) Patent Application Publication
(10) Pub. No.: US 2020/0263163 A1
(43) Pub. Date: Aug. 20, 2020

(54) MULTINOMIAL ENCODING FOR OLIGONUCLEOTIDE-DIRECTED COMBINATORIAL CHEMISTRY

(71) Applicant: Haystack Sciences Corporation, South San Francisco, CA (US)

(72) Inventors: Richard Edward Watts, South San Francisco, CA (US); Divya Kanichar, South San Francisco, CA (US)

(73) Assignee: Haystack Sciences Corporation, South San Francisco, CA (US)

(21) Appl. No.: 16/649,321

(22) PCT Filed: Sep. 24, 2018
(86) PCT No.: PCT/US18/52494

§ 371 Date: Mar. 20, 2020

Related U.S. Application Data
(60) Provisional application No. 62/562,582, filed on Sep. 25, 2017.

(51) Int. Cl. C12N 15/10; C12Q 1/6811

(52) U.S. Cl. CPC C12N 15/10; C12Q 1/6811

(57) ABSTRACT

The present disclosure relates to multifunctional molecules, including molecules according to formula (I-A) [(B1)_{M}-L₁]_{O}-G, and (I) [(B₁)_{M}-L₁]_{O}-G-[(L₂-(B₂)_{K}]_{P} wherein B₁, M, L₁, O, G, L₂, B₂, K, and P are defined herein, wherein each positional building block B1 is identified by from 1 to 5 coding regions in G, and from about 10% to 100% of the positional building blocks B₁ at position M and/or B₂ at position K, based on a total number of positional building blocks, are identified by a combination of from 2 to 5 independent coding regions. Methods of making such multifunctional molecules, and methods of serially enriching an oligonucleotide encoded library, are also disclosed. The present disclosure also relates to methods of preparing and using such multifunctional molecules to identify encoded molecules capable of binding target molecules.

### TECHNICAL FIELD

The present disclosure relates to multifunctional molecules, and multinomial methods of preparing and using such multifunctional molecules. Benefits of the methods disclosed can include reducing costs, increasing yield, and/or decreasing the time necessary to synthesize oligonucleotide encoded molecules. The present disclosure further provides methods of using multifunctional molecules to identify encoded molecules capable of binding target molecules or possessing other desirable properties like target molecule selectivity or cell permeability.

### BACKGROUND

Oligonucleotide encoded libraries can provide a useful method of directing the combinatorial synthesis of and identification of vast numbers of different molecules having different properties and reactivity. In general, an oligonucleotide encoded molecule can include an encoded portion that is tethered to an oligonucleotide portion, wherein each oligonucleotide coding region individually correlates to or identifies the structure of the encoded portion to which the oligonucleotide portion is attached. An oligonucleotide encoded library can contain millions of oligonucleotide encoded molecules, and these libraries can be subjected to assays or selection experiments designed to separate those oligonucleotide encoded molecules which possess a desired trait from those which do not. After separation, the oligonucleotide portion of the oligonucleotide encoded molecule possessing the desired trait can then be amplified by PCR (polymerase chain reaction) and sequenced using common oligonucleotide sequencing technologies. The identity of molecules possessing the desired properties can be identified or deduced by correlating the sequence information with the synthetic steps used to synthesize the encoded portion of the oligonucleotide encoded molecule.

Some methods of synthesizing oligonucleotide encoded molecules require that each synthetic step sort the multifunctional molecules into sub-pools by selectively binding a coding region of the oligonucleotide portion to an array of complementary oligonucleotides immobilized on a solid support. By selectively binding specific sequences of oligonucleotides to sequence-specific hybridization arrays, each synthetic step separates, directs, and encodes the synthetic reactions that build the encoded portion of the oligonucleotide encoded molecule. Therefore, in past methods, the number of different features in an array defines the maximum number of different chemical building blocks that can be used during each synthetic step. The traditional method of synthesizing libraries of oligonucleotide encoded molecules also requires that each feature of the sequence-specific hybridization array is exposed to the oligonucleotide portion of the multifunctional molecule being built prior to each synthetic step. This requirement imposes a strict one-to-one correlation of a coding region of the oligonucleotide portion to the building block being added to the encoded portion. A benefit of this requirement has included a one-to-one deduction or identification of synthetic steps used to encode the encoded portion of the oligonucleotide encoded molecule. Another benefit is that the higher the number of different coding regions in the oligonucleotide portion, the higher the number of different building block combinations that can be reacted to build the encoded portion.

### SUMMARY

As recognized herein, the traditional method of synthesizing oligonucleotide encoded molecules suffers from several drawbacks. First, the number of possible different oligonucleotide encoded molecules in a library increases with the number of different features on the sequence-specific hybridization array. Therefore, the greater the number of different oligonucleotide encoded molecules desired, the greater the number of expensive oligonucleotides that must be purchased and immobilized to form the sequence-specific hybridization array. This expense can become a significant burden.

To achieve adequate yield of oligonucleotide encoded molecules from a diverse library of millions of different molecules, every member of the library must be given sufficient time and proximity to each feature of the array to achieve accurate sorting. For example, there may only be one coding region on one molecule capable of reacting with one feature of the hybridization array such that it becomes critical to ensure that such a molecule has sufficient opportunity to react with that one feature. This typically requires flowing or soaking the entire library of molecules in solution over each feature of the hybridization array in series. This method imposes impractically high processing times. Additionally, because the coding regions on the oligonucleotide are simultaneously exposed to a great number of different oligonucleotides immobilized on the hybridization array, the possibility of cross/mis-hybridization and incorrect sorting is significant.

By way of example, suppose a hybridization array has two features, and a library has two coding regions. If half the library solution is passed through one feature, while simultaneously half the library solution is passed through the second feature, then half the library will be captured—half the library DNA passing through a feature will have the correct sequence, half will not. Further, suppose the solution passing through both features is collected in the same vial, then split, and half is flowed through the first feature and half through the second feature. Again, half of the remaining DNA will be captured. Each time the solution is collected in the same vial, mixed, and flowed through the two features, another half of the remaining DNA will be captured. Under the same regime with 4 features and 4 coding regions, only ¼ of all DNA will be captured at each pass. If there are 384 features and 384 coding regions, only 1/384th of the remaining DNA will be captured at each pass. Thus, simultaneous exposure of the library to multiple features of a hybridization array is a recipe for slow sorting, low yield, and inaccurate synthesis.

Consider a second example: the half of the solution that is flowed through the first feature is independently flowed through the second feature. Then, the half of the solution flowed through the second feature is independently flowed through the first feature. This sequential method would capture all of the coding region of oligonucleotides with just two operations. However, the greater the number of features, the less practical the method of sequential processing becomes. For example, while it may be possible to pipette by hand through 384 features of an array, one feature at a time, evaporation due to handling, leaks, spills, and mistakes reduce the efficiency and practicality of this method, or engender the need for expensive instrumentation.

Recognized herein is thus a need for a more efficient method of synthesizing oligonucleotide encoded molecules having lower costs and/or shorter processing times while maintaining or providing higher yields, greater simplicity, and more accurate synthesis.

The present disclosure relates to methods and molecules of multinomial oligonucleotide directed and recorded combinatorial synthesis of multifunctional molecules with such features as improved yield, improved sorting fidelity, and improved specificity of building block encoding. In certain embodiments, the multifunctional molecules are molecules according to formula (I),

[(B₁)_{M}-L₁]_{O}-G-[(L₂-(B₂)_{K}]_{P} (I)

wherein

G includes an oligonucleotide, the oligonucleotide comprising at least two coding regions, wherein the at least two coding regions are single stranded;

B₁ is a positional building block and M represents an integer from 1 to 20;

B₂ is a positional building block and K represents an integer from 1 to 20, wherein B₁ and B₂ are the same or different, wherein M and K are the same or different;

L₁ is a linker that operatively links B₁ to G;

L₂ is a linker that operatively links B₂ to G;

O is zero or 1;

P is zero or 1;

provided that at least one of O and P is 1; and

wherein each positional building block B₁ at position M and/or B₂ at position K is identified by from 1 to 5 coding regions, and from about 10% to 100% of the positional building blocks B₁ at position M and/or B₂ at position K, based on a total number of positional building blocks, are identified by a combination of from 2 to 5 independent coding regions.

In certain embodiments of the molecule of formula (I), G includes a sequence represented by the formula (C_{N}-(Z_{N}-C_{N+1})_{A}) or (Z_{N}-(C_{N}-Z_{N+1})_{A}), wherein C is a coding region, Z is a non-coding region, N is an integer from 1 to 20, and A is an integer from 1 to 20; wherein each non-coding region contains from 0 to 50 nucleotides and is optionally double stranded. In certain embodiments of the molecule of formula (I), each coding region contains from 6 to 50 nucleotides. In certain embodiments of the molecule of formula (I), each coding region contains from 8 to 30 nucleotides. In certain embodiments of the molecule of formula (I), at least one of O or P is zero. In certain embodiments of the molecule of formula (I), from about 20% to 100% of the positional building blocks B₁ at position M and/or B₂ at position K, based on the total number of positional building blocks, are identified by a combination of from 2 to 5 independent coding regions. In certain embodiments of the molecule of formula (I), from about 20% to 100% of the positional building blocks B₁ at position M and/or B₂ at position K, based on the total number of positional building blocks, are identified by a combination of from 2 to 3 independent coding regions. In certain embodiments of the molecule of formula (I), P is 0; O is 1; and from about 30% to 100% of the positional building blocks B₁ at position M, based on the total number of positional building blocks, are identified by a combination of from 2 to 3 independent coding regions. In certain embodiments of the molecule of formula (I), O is 0; P is 1; and from about 30% to 100% of the positional building blocks B₂ at position K, based on the total number of positional building blocks, are identified by a combination of from 2 to 3 independent coding regions.

A method of identifying probe molecules capable of binding or selecting for a target molecule is disclosed. In certain embodiments of the method of identifying probe molecules, the method includes,

exposing the target molecule to a pool of probe molecules, wherein the probe molecules are according to formula (I), formula (III), and/or formula (IV),

removing at least one probe molecule that does not bind the target molecule,

amplifying the oligonucleotide of G from the at least one probe molecule that was not removed from the target molecule to form a copy sequence,

sequencing the copy sequence to identify each coding region and combination of coding regions of the probe molecule to further identify each positional building block B₁ at position M and/or B₂ at position K. In certain embodiments of the method of identifying probe molecules, the method includes sequencing the copy sequence to identify each coding region and combination of from 2 to 3 independent coding regions of the probe molecule to further identify at least one of each positional building block B₁ at position M and B₂ at position K.

A method of forming a molecule of formula (I) is disclosed herein. In certain embodiments of the method of forming a molecule of formula (I), the method includes,

providing at least one first hybridization array, the at least one first hybridization array comprising at least one first single stranded anti-codon oligomer immobilized on the at least one first hybridization array, wherein the at least one first single stranded anti-codon oligomer immobilized on the at least one first hybridization array is capable of hybridizing to a first coding region of a molecule of formula (II):

[(B₁)_{(M-1)}-L₁]_{O}-G-[(L₂-(B₂)_{(K-1)})_{P} (II)

wherein

G includes an oligonucleotide, the oligonucleotide comprising at least two coding regions, wherein the at least two coding regions are single stranded;

B₁ is a positional building block and M represents an integer from 1 to 20; B₂ is a positional building block and K represents an integer from 1 to 20, wherein

B₁ and B₂ are the same or different, wherein M and K are the same or different;

L₁ is a linker that operatively links B₁ to G;

L₂ is a linker that operatively links B₂ to G;

O is zero or 1;

P is zero or 1;

provided that at least one of O and P is 1; and

wherein each positional building block B₁ at position M and/or B₂ at position K is identified by from 1 to 5 coding regions, and from about 10% to 100% of the positional building blocks B₁ at position M and/or B₂ at position K, based on a total number of positional building blocks, are identified by a combination of from 2 to 5 independent coding regions;

sorting the pool of molecules of formula (II) into a first set of sub-pools by hybridizing the first coding region of the molecules of formula (II) to the at least one first single stranded anti-codon oligomer immobilized on the at least one first hybridization array;

releasing the first set of sub-pools of molecules of formula (II) from the at least one first hybridization array into separate containers;

providing at least one second hybridization array, the at least one second hybridization array comprising at least one second single stranded anti-codon oligomer immobilized on the at least one second hybridization array, wherein the at least one second single stranded anti-codon oligomer immobilized on the at least one second hybridization array is capable of hybridizing to a second coding region of a molecule of formula (II):

independently sorting each, or at least one, of the first set of sub-pools of molecules of formula (II) into a second set of sub-pools by hybridizing the second coding region of the molecules of formula (II) to the at least one second single-stranded anti-codon oligomer immobilized on the at least one second hybridization array;

providing at least one of building block B₁ and B₂; and

reacting the at least one of building block B₁ and B₂ with the molecule of formula (II) to form a sub-pool of molecules of formula (I):

[(B₁)_{M}-L₁]_{O}-G-[(L₂-(B₂)_{K})_{P} (I)

wherein

G includes an oligonucleotide, the oligonucleotide comprising at least two coding regions, wherein the at least two coding regions are single stranded;

B₁ is a positional building block and M represents an integer from 1 to 20;

B₂ is a positional building block and K represents an integer from 1 to 20, wherein B₁ and B₂ are the same or different, wherein M and K are the same or different;

L₁ is a linker that operatively links B₁ to G;

L₂ is a linker that operatively links B₂ to G;

O is zero or 1;

P is zero or 1;

provided that at least one of O and P is 1; and

wherein each positional building block B₁ at position M and/or B₂ at position K is identified by from 1 to 5 coding regions, and from about 10% to 100% of the positional building blocks B₁ at position M and/or B₂ at position K, based on a total number of positional building blocks, are identified by a combination of from 2 to 5 independent coding regions.

In certain embodiments of the method of forming a molecule of formula (I), the method further includes, before the step of the reaction step, (a) releasing the second set of sub-pool of molecules of formula (II) from the at least one second hybridization array into a second set of separate containers; (b) providing at least one third hybridization array, the at least one third hybridization array comprising at least one third single stranded anti-codon oligomer immobilized on the at least one third hybridization array, wherein the at least one third single stranded anti-codon oligomer immobilized on the at least one third hybridization array is capable of hybridizing to a third coding region of a molecule of formula (II); (c) independently sorting at least one sub-pool from the second set of sub-pools of molecules of formula (II) into a third set of sub-pools by hybridizing the third coding region of the third set of sub-pools of molecules of formula (II) to the at least one third single stranded anti-codon oligomer immobilized on the at least one third second hybridization array; and optionally, repeating steps (a), (b), and (c). In certain embodiments of the method of forming a molecule of formula (I), each coding region contains from 6 to 50 nucleotides. In certain embodiments of the method of forming a molecule of formula (I), each coding region contains from 8 to 30 nucleotides. In certain embodiments of the method of forming a molecule of formula (I), at least one of O or P is zero. In certain embodiments of the method of forming a molecule of formula (I), from about 20% to 100% of the positional building blocks B₁ at position M and/or B₂ at position K, based on a total number of positional building blocks, are identified by a combination of from 2 to 5 independent coding regions. In certain embodiments of the method of forming a molecule of formula (I), from about 20% to 100% of the positional building blocks B₁ at position M and/or B₂ at position K, based on a total number of positional building blocks, are identified by a combination of from 2 to 3 independent coding regions. In certain embodiments of the method of forming a molecule of formula (I), P is 0; O is 1; and from about 30% to 100% of the positional building blocks B₁ at position M, based on the total number of positional building blocks, are identified by a combination of from 2 to 3 independent coding regions. In certain embodiments of the method of forming a molecule of formula (I), O is 0; P is 1; and from about 30% to 100% of the positional building blocks B₂ at position K, based on the total number of positional building blocks, are identified by a combination of from 2 to 3 independent coding regions.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown.

FIG. 1 is an illustration of an embodiment of a method that uses a single coding region to direct of a step of synthesizing a multifunctional molecule.

FIG. 2 is a flow diagram which illustrates two steps of using a single coding region to direct the synthesis of a multifunctional molecule.

FIG. 3 is an illustration of an embodiment of a method of uses a combination of two coding regions to direct of a step of synthesizing a multifunctional molecule.

FIG. 4 is a flow diagram which illustrates two steps of using a combination of two coding regions to direct the synthesis of a multifunctional molecule.

FIG. 5 is a photograph of a gel electrophoresis experiment, wherein embodiments of the molecule are digested and separated to determine if enrichment based on specific hybridization is occurring.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one of H₁ and H₂” means H₁, H₂, or both.

Unless otherwise noted, the term “about” refers to ±10% of the non-percentage number that is described, rounded to the nearest whole integer. For example, about 100 mm, would include 90 to 110 mm. Unless otherwise noted, the term “about” refers to ±5% of a percentage number. For example, about 20% would include 15 to 25%. When the term “about” is discussed in terms of a range, then the term refers to the appropriate amount less than the lower limit and more than the upper limit. For example, from about 100 to about 200 mm would include from 90 to 220 mm.

Unless otherwise noted, the term “hybridize,” “hybridizing,” “hybridized,” and “hybridization” includes Watson-Crick base pairing, which includes guanine-cytosine and adenine-thymine (G-C and A-T) pairing for DNA and guanine-cytosine and adenine-uracil (G-C and A-U) pairing for RNA. Typically, these terms are used in the context of the selective recognition of a strand of nucleotides for a complementary strand of nucleotides, called an anti-codon or anti-coding region.

The terms “selectively hybridizing,” “selective hybridization,” “selectively sorting,” and “selective recognition” refer to a selectivity of from 3:1 to 100:1 or more of a complementary oligonucleotide strand relative to a non-complementary oligonucleotide strand.

The term “multifunctional molecule” refers to a molecule of the present disclosure that contains an oligonucleotide and at least one encoded portion.

The term “encoded portion” refers to one or more parts of the multifunctional molecule that only contain building blocks, such as positional building blocks B₁ and B₂. The term “encoded portion” does not include, for example, a linker, even though these structures may be added as part of the process of synthesizing the encoded portion.

The term “encoded molecule” refers to a molecule that would be or is formed if the encoded portion of the multifunctional molecule were removed or separated from the rest of the multifunctional molecule.

The term “probe molecule” refers to a molecule that is used to determine which encoded portion of a multifunctional molecule or encoded molecule is capable of binding a target molecule or selecting for desirable properties like target molecule selectivity or cell permeability. The term “probe molecule” can include a multifunctional molecule.

The term “target molecule” refers to a molecule or structure. For example, structures include multi-macromolecular complexes, such as ribosomes, and liposomes.

The term “encoded probe molecule” is used interchangeably with the term multifunctional molecule.

The phrase “total number of positional building blocks” refers to an aggregate number of building blocks in each encoded portion present.

The term “identified,” identify, or “identifies” refers to a correlation present between a coding region or a combination of coding regions of the oligonucleotide portion and the structure and/or sequence of building blocks of the encoded portion of a multifunctional molecule. This correlation of sequence of a coding region can be combined with the knowledge of the synthetic steps used to construct the encoded portion to allow for the deduction or identification of the structure, predicted structure, and/or sequence of the encoded portion, even if the sequence is indirectly obtained from a PCR generated copy of the multifunctional molecule.

The terms “first,” “second,” etc. are understood to be terms that merely designate or distinguish which object is being referred to, and are often based on a sequence of whichever one happens to be encountered first. For example, “first” array is an array used prior to a “second” array, and a first coding region is the coding region that happens to be capable of being immobilized on the first array. Unless otherwise noted, the terms “first,” “second,” etc. do not refer to a position within the DNA strand molecule. For example, it is understood that a first coding region and a second coding region may or may not be sequential and may or may not be close to one another within the oligonucleotide portion.

In the present disclosure, the hyphen or dashes in a molecular formula indicate that the parts of the formula are directly connected to each other through a covalent bond or hybridization.

Unless otherwise noted, all ranges of nucleotides, integer values, and percentages include all intermediate integer numbers as well as the endpoints. For example, the range of from 5 to 10 oligonucleotides would be understood to include 5, 6, 7, 8, 9, and 10 nucleotides.

In certain embodiments, the present disclosure relates to multifunctional molecules that contain at least one oligonucleotide portion and at least one encoded portion, wherein the oligonucleotide portion directed or encoded the synthesis of the at least one encoded portion using combinatorial chemistry. In certain embodiments, the oligonucleotide portion of the multifunctional molecule can identify or facilitate the deduction of the at least one encoded portion of the multifunctional molecule. In certain embodiments, a multifunctional molecule of the present disclosure contains at least one oligonucleotide or oligonucleotide portion that contains at least two coding regions, wherein a combination of the at least two coding regions corresponds to and can be used to identify or deduce the sequence of building blocks in or structure of the encoded portion. In certain embodiments, the at least one oligonucleotide or oligonucleotide portion can be amplified by PCR to produce copies of the at least one oligonucleotide or oligonucleotide portion and the original or copies can be sequenced to determine the identity of a combination of at least two coding regions of the multifunctional molecule. In certain embodiments, the identity of the combination of the at least two coding regions can be correlated to the series of combinatorial chemistry steps used to synthesize the encoded portion of the multifunctional molecule to which the PCR copy corresponds.

In certain embodiments, the present disclosure also relates to methods of forming multifunctional molecules, and to methods of exposing target molecules to the multifunctional molecules to identify or facilitate the deduction of which encoded portion, and therefore which encoded molecule, exhibits a desired property, including but not limited to the capability of binding a target molecule or molecules, of not binding other anti-target molecules, of being resistant to chemical changes made by enzymes, of being readily chemically changed by enzymes, of having degrees of water solubility, of being tissue permeable, and of being cell-permeable.

In certain embodiments, a benefit of using a combination of two or more coding regions to direct the synthesis of or encode for a building block can include that many of the sorting tasks are greatly reduced. For example, if hybridization arrays are used in sequence, rather than in massive parallel, then fewer oligonucleotides can be used to achieve selective separation during or prior to a synthetic step. Similarly, if hybridization arrays are used in sequence, rather than massive parallel, then the oligonucleotides on a hybridization array can be designed to possess sufficient dissimilarity of sequence that mis-hybridizations are minimized or eliminated.

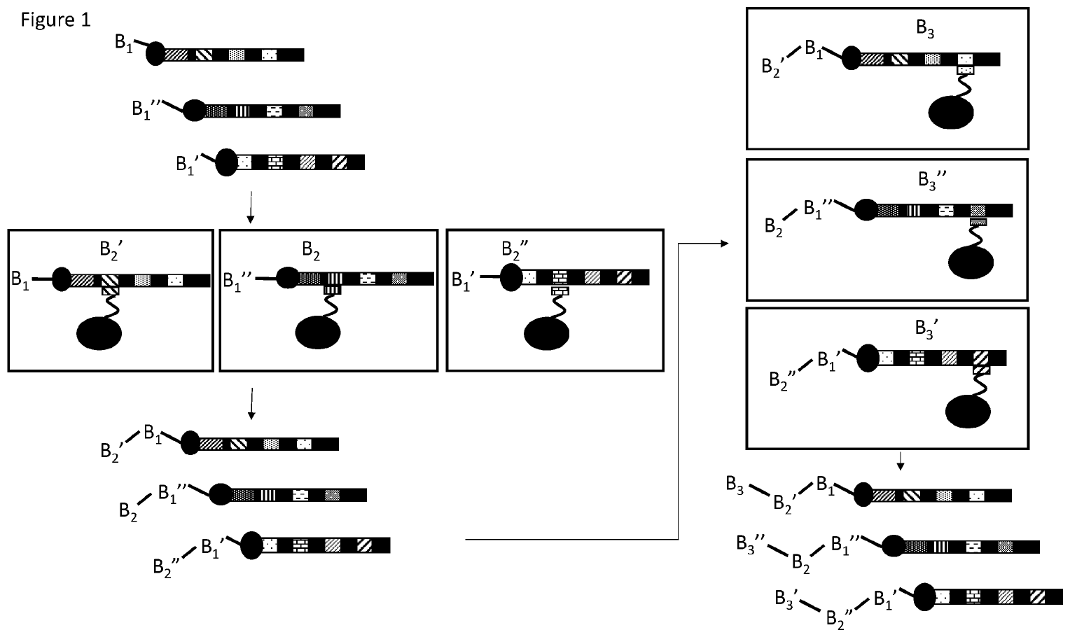

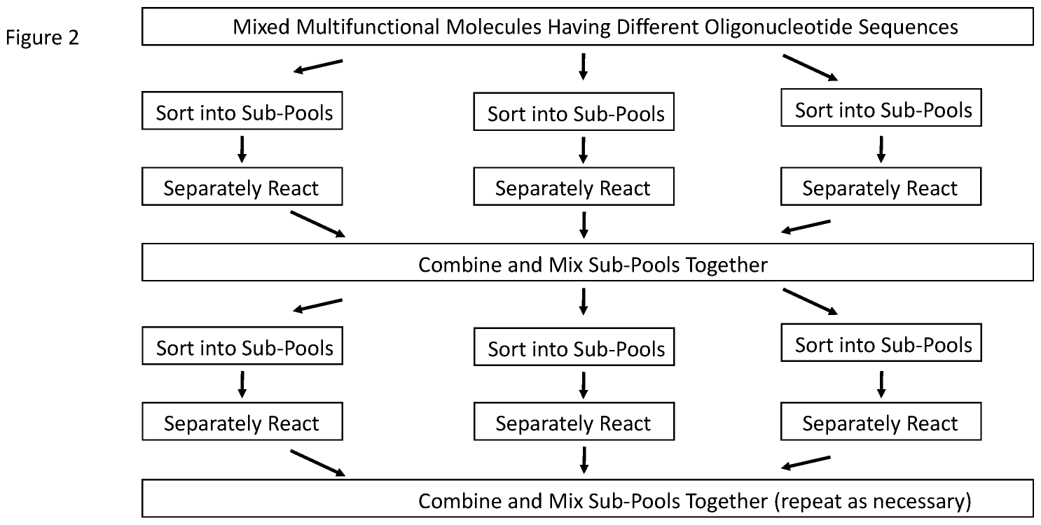

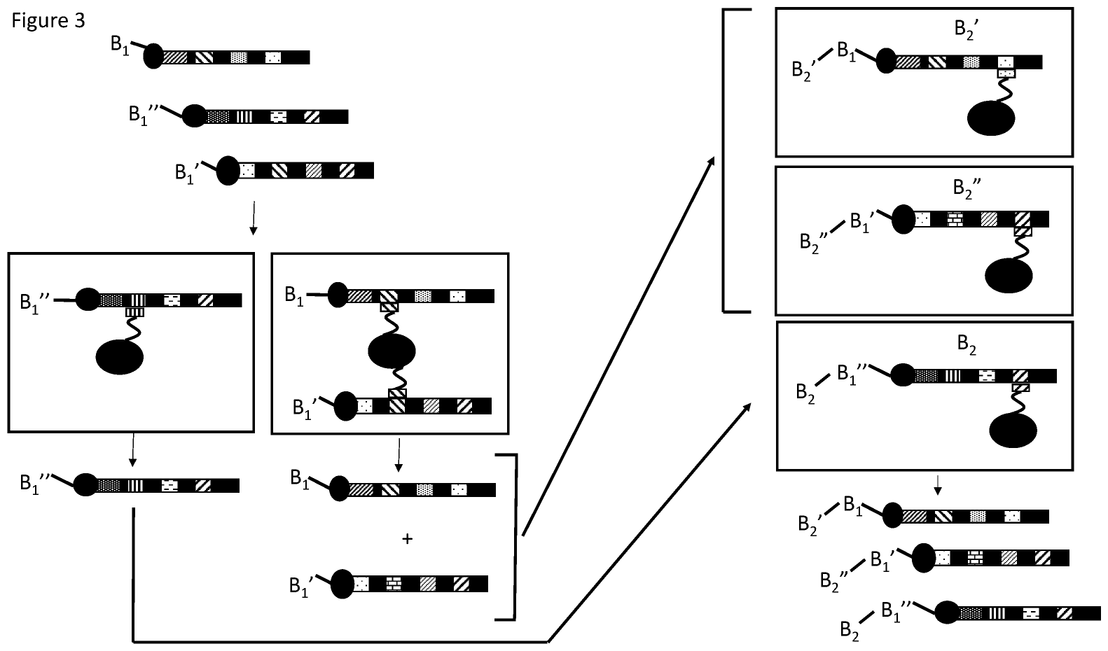

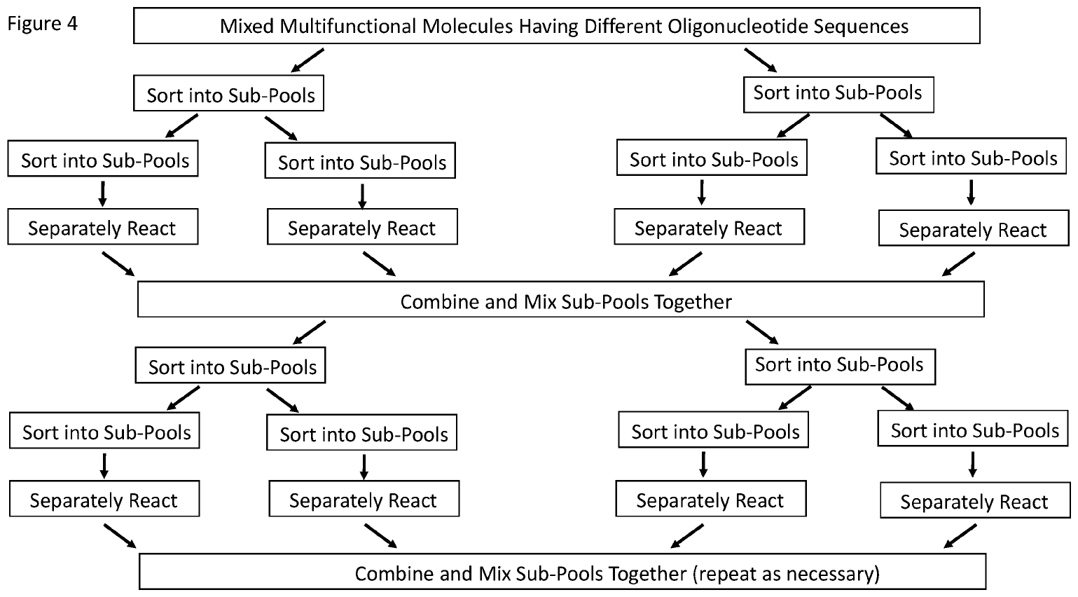

Referring to FIGS. 1 and 2, under the previous oligonucleotide directed or encoded synthesis pioneered by the present inventor, the synthetic process could be described as a “split, react, mix process,” where the splitting step required a massively parallel hybridization array. Referring to FIGS. 3 and 4, in an embodiment, the presently disclosed process could be described as a sequence of “split, split, react, mix process” or “(split)²⁻⁵, react, mix process,” where the number of splits is two or more, usually from 2 to 5, and the splitting or sorting step can use arrays with smaller numbers of features, and therefore fewer oligonucleotide strands in the hybridization array and/or the encoding portion of the multifunctional molecule. This discovery is highly counterintuitive because so much of high-through put processing in, for example, the semiconductor industry or the genome sequencing industry, is based on larger and larger parallel processing to reduce processing time and costs. However, due to the unique selectivity requirements imposed by oligonucleotide hybridization, it has been discovered that the process of the present disclosure can greatly improve the efficient and accurate synthesis of multifunctional molecules and probe molecules by introducing a few sequential sorting steps into an otherwise parallel process.

By way of example, in order to sort 384 different sequences, the traditional way of synthesizing oligonucleotide encoded molecules, discussed in the introduction section above, would could code for 384 features, but would impose a yield of less than 1/384th or a series of 384 sorting steps. In contrast, a 384 feature library processed by an embodiment of the presently disclosed process can sort a library on a large scale array of 16 features. Then each of the 16 sub-pools can be sorted on 24 identical small scale arrays in parallel. In this manner, 40 different oligonucleotides can encode 384 different building blocks. These oligonucleotides can effectively encode 384 building blocks, even though fewer sequences were used. One benefit of the presently disclosed method is that the cost of synthesis is drastically reduced, because, in practice, it is vastly more cost effective to buy 20 nanomoles of each of 40 oligonucleotides (“oligos”) than it is to buy 1 nanomole of each of 384 modified oligos.

In certain embodiments, the molecule of formula (I) is a multifunctional molecule. In certain embodiments of the molecule of formula (I), G includes an oligonucleotide that directed or selected for the synthesis of the encoded portion. In certain embodiments of the molecule of formula (I), (B₁)_{M} and (B₂)_{K} each represent an encoded portion. In certain embodiments of the molecule of formula (I), the molecule contains an oligonucleotide portion and at least one encoded portion. It is understood that many of the structural features of the oligonucleotide in G are discussed herein in terms of their having directed or encoded the synthesis of the at least one encoded portion of the molecule of formula (I) as well as the molecular structural relationship or correlation that this synthetic process imposes on the structure of the multifunctional molecule. It is understood that many of the structural features of the oligonucleotide in G of the molecule of formula (I) are discussed in terms of the ability of the oligonucleotide in G, or a PCR copy thereof, to identify, correlate, or facilitate the deduction of the synthesis steps used to prepare the molecule of formula (I). Therefore, it is understood that there is a correlation between the sequence and/or structure of the building blocks of the encoded portion and the sequence or combination of sequences of the coding regions of the oligonucleotide portion.

In certain embodiments of the molecule of formula (I), G includes or is an oligonucleotide. In certain embodiments, the oligonucleotide contains at least two coding regions, wherein from 1% to 100%, including from about 50% to 100%, including from about 90% to 100%, of the coding regions are single stranded. In certain embodiments, the oligonucleotide in G contains at least one terminal coding region, wherein one or two of the terminal coding regions are single stranded. In certain embodiments, the oligonucleotide in G contains at least one terminal coding region, wherein one or two of the terminal coding regions are double stranded.

In certain embodiments of the molecule of formula (I), G can include a hairpin structure comprising oligonucleotides. In certain embodiments, G does not include a hairpin structure, such as in formula (III) and formula (IV), as discussed below. The term “hairpin structure” as used in the present disclosure refers to a molecular structure that contains from 60% to 100% nucleotides by mass percent, and can hybridize to a terminal coding region of the oligonucleotide G, or comprises a terminal coding region in G. In certain embodiments of the hairpin structure, the hairpin structure forms a single, continuous polymer chain, and contains at least one overlapping portion (commonly called a “stem”), wherein the overlapping portion contains a sequence of nucleotides that is hybridized to a complementary sequence of the same hairpin structure. In certain embodiments of the hairpin structure, a bridge structure connects two separate oligonucleotide strands; said bridge structure may be comprised of a polyethylene glycol (PEG) polymer of between 2 and 20 PEG units, including between 3 and 15 PEG units, including between 6 and 12 PEG units. In certain embodiments of the hairpin structure, the bridge structure may be comprised of an alkane chain of up to 30 carbons, or a polyglycine chain of up to 20 units, or comprised of some other chain that bears a reactive functional group.

In certain embodiments of the molecule of formula (I), the oligonucleotide in G contains at least two coding regions, including from 2 to about 21 coding regions, including from 3 to 10 coding regions, including from 3 to 5 coding regions. In certain embodiments, if the number of coding regions falls below 2, then no combination of the coding regions would be possible. In certain embodiments, if the number of coding regions exceeds 20, then synthetic inefficiencies would interfere with accurate synthesis.

In certain embodiments of the molecule of formula (I), from about 50% to 100% of the at least two coding regions contain from about 6 to about 50 nucleotides, including from about 12 to about 40 nucleotides, including from about 8 to about 30 nucleotides. In certain embodiments, if the coding region contains less than about 6 nucleotides then the coding region cannot accurately direct synthesis of the encoded portion. In certain embodiments, if the coding region contains more than about 50 nucleotides then the coding region could become cross reactive. Such cross reactivity would interfere with the ability of the coding regions to accurately direct and identify the synthesis steps used to synthesize the encoded portion of a molecule of formula (I).

In certain embodiments of the molecule of formula (I), a purpose of the oligonucleotide in G is to direct the synthesis of at least one encoded portion of the molecule of formula (I) by selectively hybridizing to a complementary anti-coding strand. In certain embodiments, the coding regions are single stranded to facilitate hybridization with a complementary strand. In certain embodiments, from 70% to 100%, including from 80% to 99%, including from 80 to 95%, of the coding regions are single stranded. It is understood that the complementary strand for a coding region, if present, could be added after steps of encoding the encoded portion of the molecule of formula (I) during synthesis.

In certain embodiments, the oligonucleotide can contain natural and unnatural nucleotides. Suitable nucleotides include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Other suitable bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages. It is understood that an oligonucleotide is a polymer of nucleotides. The terms “polymer” and “oligomer” are used herein interchangeably. In certain embodiments, the oligonucleotide does not have to contain contiguous bases. In certain embodiments, the oligonucleotide can be interspersed with linker moieties or non-nucleotide molecules.

In certain embodiments of the molecule of formula (I), the oligonucleotide in G contains from about 60% to 100%, including from about 80% to 99%, including from about 80% to 95% DNA nucleotides. In certain embodiments, the oligonucleotide contains from about 60% to 100%, including from about 80% to 99%, including from about 80% to 95% RNA nucleotides.

In certain embodiments of the molecule of formula (I), the oligonucleotide in G contains at least two coding regions, wherein the at least two of the coding regions overlap so as to be coextensive, provided that the overlapping coding regions only share from about 30% to 1% of the same nucleotides, including about 20% to 1%, including from about 10% to 2%. In certain embodiments of the molecule of formula (I), the oligonucleotide in G is from about 30% to 100%, including about from 60% to 100%, including about from 80% to 100%, single stranded. In certain embodiments of the molecule of formula (I), the oligonucleotide in G contains at least two coding regions, wherein at least two of the coding regions are adjacent. In certain embodiments of the molecule of formula (I), the oligonucleotide in G contains at least two coding regions, wherein the at least two coding regions are separated by regions of nucleotides that do not direct or record synthesis of an encoded portion of the molecule of formula (I).

The term “non-coding region,” when present, refers to a region of the oligonucleotide that either cannot hybridize with a complementary strand of nucleotides to direct the synthesis of the encoded portion of the molecule of formula (I) or does not correspond to any anti-coding oligonucleotide used to sort the molecules of formula (I) during synthesis. In certain embodiments, non-coding regions are optional. In certain embodiments, the oligonucleotide contains from 1 to about 20 non-coding regions, including from 2 to about 9 non-coding regions, including from 2 to about 4 non-coding regions. In certain embodiments, the non-coding regions contain from about 4 to about 50 nucleotides, including from about 12 to about 40 nucleotides, and including from about 8 to about 30 nucleotides.

In certain embodiments of the molecule of formula (I), one purpose of the non-coding regions is to separate coding regions to avoid or reduce cross-hybridization, because cross-hybridization would interfere with accurate encoding of the encoded portion of the molecule of formula (I). In certain embodiments, one purpose of the non-coding regions is to add functionality, other than just hybridization or encoding, to the molecule formula (I). In certain embodiments, one or more of the non-coding regions can be a region of the oligonucleotide that is modified with a label, such as a fluorescent label or a radioactive label. Such labels can facilitate the visualization or quantification of molecules for formula (I). In certain embodiments, one or more of the non-coding regions are modified with a functional group or tether which facilitates processing. In certain embodiments, one or more of the non-coding regions are double stranded, which reduces cross-hybridization. In certain embodiments, it is understood that non-coding regions are optional. In certain embodiments, suitable non-coding regions do not interfere with PCR amplification of the oligonucleotide.

In certain embodiments, one or more of the coding regions can be a region of the oligonucleotide in G that is modified with a label, such as a fluorescent label or a radioactive label. Such labels can facilitate the visualization or quantification of molecules for formula (I). In certain embodiments, one or more of the coding regions are modified with a functional group or tether which facilitates processing.

In certain embodiments of the molecule of formula (I), G comprises a sequence represented by the formula (C_{N}-(Z_{N}-C_{N+1})_{A}) or (Z_{N}-(C_{N}-Z_{N+1})_{A}), wherein C is a coding region, Z is a non-coding region, N is an integer from 1 to 20, and A is an integer from 1 to 20; wherein each non-coding region contains from 0 to 50 nucleotides and is optionally double stranded. In certain embodiments of the molecule of formula (I), each or most of the coding regions contains from 6 to 50 nucleotides. In certain embodiments of the molecule of formula (I), each or most of the coding regions contain from 8 to 30 nucleotides.

In certain embodiments of the molecule of formula (I), from about 10% to 100% of the positional building blocks B₁ at position M and/or B₂ at position K correlate to a combination of from 2, 3, 4, or 5 coding regions, including from about 20% to 100%, including from about 30% to 100%, including from about 50% to 100%, including from about 70% to 100%, including from about 90% to 100%. Conversely, in certain embodiments of the molecule of formula (I), from 0 to about 90% of the positional building blocks B₁ at position M and/or B₂ at position K correlate to or are identified by a single coding region, including from 0 to about 10%, including from 0 to about 20%, including from 0 to about 30%, including from 0 to about 50%, including from 0 to about 70%.

In certain embodiments of the molecule for formula (I), B represents a positional building block. The phrase “positional building block” as used in the present disclosure means one unit in a series of individual building block units bound together as subunits forming a larger molecule molecular structure. In certain embodiments, (B₁)_{M} and (B₂)_{K} each independently represents a series of individual building block units bound together to form a polymer chain having M and K number of units, respectively. For example, wherein M is 10, then (B)₁₀, refers to a chain of building block units: B₁₀-B₉-B₈-B₇-B₆-B₅-B₄-B₃-B₂-B₁. For example, where M is 3 and K is 2, then formula (I) can accurately be represented by the following formula:

[((B₁)₃-(B₁)₂-(B₁)₁-L₁]_{O}-G-[(L₂-(B₂)₁-(B₂)₂]_{P}.

It is understood M and K each independently serve as a positional identifier for each individual unit of B, and that the “1” or “2” of B₁ or B₂ merely serves to distinguish which chain is being referred to.

The precise definition of the term “building block” in the present disclosure depends on its context. A “building block” is a chemical structural unit capable of being chemically linked to other chemical structural units. In certain embodiments, a building block has one, two, or more reactive chemical groups that allow the building block to undergo a chemical reaction that links the building block to other chemical structural units. It is understood that part or all of the reactive chemical group of a building block may be lost when the building block undergoes a reaction to form a chemical linkage. For example, a building block in solution may have two reactive chemical groups. In this example, the building block in solution can be reacted with the reactive chemical group of a building block that is part of a chain of building blocks to increase the length of a chain, or extend a branch from the chain. When a building block is referred to in the context of a solution or as a reactant, then the building block will be understood to contain at least one reactive chemical group, but may contain two or more reactive chemical groups. When a building block is referred to the in the context of a polymer, oligomer, or molecule larger than the building block by itself, then the building block will be understood to have the structure of the building block as a (monomeric) unit of a larger molecule, even though one or more of the chemical reactive groups will have been reacted.

The types of molecule or compound that can be used as a building block are not generally limited, so long as one building block is capable of reacting together with another building block to form a covalent bond. In certain embodiments, a building block has one chemical reactive group to serve as a terminal unit. In certain embodiments, a building block has 1, 2, 3, 4, 5, or 6 suitable reactive chemical groups. In certain embodiments, the positional building blocks of B each independently have 1, 2, 3, 4, 5, or 6 suitable reactive chemical groups. Suitable reactive chemical groups for building blocks include, a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a haloacetate, an aryl halide, an azide, a halide, a triflate, a diene, a dienophile, a boronic acid, an alkyne, and an alkene.

Any coupling chemistry can be used to connect building blocks, provided that the coupling chemistry is compatible with the presence of an oligonucleotide. Exemplary coupling chemistry includes, formation of amides by reaction of an amine, such as a DNA-linked amine, with an Fmoc-protected amino acid or other variously substituted carboxylic acids; formation of ureas by reaction of an amine, including a DNA-linked amine, with an isocyanate and another amine (ureation); formation of a carbamate by reaction of amine, including a DNA-linked amine, with a chloroformate (carbamoylation) and an alcohol; formation of a sulfonamide by reaction of an amine, including a DNA-linked amine, with a sulfonyl chloride; formation of a thiourea by reaction of an amine, including a DNA-linked amine, with thionocarbonate and another amine (thioureation); formation of an aniline by reaction of an amine, including a DNA-linked amine, with a heteroaryl halide (SNAr); formation of a secondary amine by reaction of an amine, including a DNA-linked amine, with an aldehyde followed by reduction (reductive amination); formation of a peptoid by acylation of an amine, including a DNA-linked amine, with chloroacetate followed by chloride displacement with another amine (an SN2 reaction); formation of an alkyne containing compound by acylation of an amine, including a DNA-linked amine, with a carboxylic acid substituted with an aryl halide, followed by displacement of the halide by a substituted alkyne (a Sonogashira reaction); formation of a biaryl compound by acylation of an amine, including a DNA-linked amine, with a carboxylic acid substituted with an aryl halide, followed by displacement of the halide by a substituted boronic acid (a Suzuki reaction); formation of a substituted triazine by reaction of an amine, including a DNA-linked amine, with a cyanuric chloride followed by reaction with another amine, a phenol, or a thiol (cyanurylation, Aromatic Substitution); formation of secondary amines by acylation of an amine including a DNA-linked amine, with a carboxylic acid substituted with a suitable leaving group like a halide or triflate, followed by displacement of the leaving group with another amine (SN2/SN1 reaction); and formation of cyclic compounds by substituting an amine with a compound bearing an alkene or alkyne and reacting the product with an azide, or alkene (Diehls-Alder and Huisgen reactions). In certain embodiments of the reactions, the molecule reacting with the amine group, including a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a chloroacetate, an aryl halide, an alkene, halides, a boronic acid, an alkyne, and an alkene, has a molecular weight of from about 30 to about 330 Daltons.

In certain embodiments of the coupling reaction, a first building block might be added by substituting an amine, including a DNA-linked amine, using any of the chemistries above with molecules bearing secondary reactive groups like amines, thiols, halides, boronic acids, alkynes, or alkenes. However, it is understood that this step is not limited to the chemistries above. Then the secondary reactive groups can be reacted with building blocks bearing appropriate reactive groups. Exemplary secondary reactive group coupling chemistries include, acylation of the amine, including a DNA-linked amine, with an Fmoc-amino acid followed by removal of the protecting group and reductive amination of the newly deprotected amine with an aldehyde and a borohydride; reductive amination of the amine, including a DNA-linked amine, with an aldehyde and a borohydride followed by reaction of the now-substituted amine with cyanuric chloride, followed by displacement of another chloride from triazine with a thiol, phenol, or another amine; acylation of the amine, including a DNA-linked amine, with a carboxylic acid substituted by a heteroaryl halide followed by an SNAr reaction with another amine or thiol to displace the halide and form an aniline or thioether; and acylation of the amine, including a DNA-linked amine, with a carboxylic acid substituted by a haloaromatic group followed by substitution of the halide by an alkyne in a Sonogashira reaction; or substitution of the halide by an aryl group in a boronic ester-mediated Suzuki reaction.

In certain embodiments, the coupling chemistries are based on suitable bond-forming reactions, such as those described in, for example, March, Advanced Organic Chemistry, fourth edition, New York: John Wiley and Sons (1992), Chapters 10 to 16; Carey and Sundberg, Advanced Organic Chemistry, Part B, Plenum (1990), Chapters 1-11; and Coltman et al., Principles and Applications of Organotransition Metal Chemistry, University Science Books, Mill Valley, Calif. (1987), Chapters 13 to 20; each of which is incorporated herein by reference in its entirety.

In certain embodiments, a building block can include one or more functional groups in addition to the reactive group or groups employed to attach a building block. One or more of these additional functional groups can be protected to prevent undesired reactions of these functional groups. Suitable protecting groups for a variety of functional groups may be used (e.g., Greene and Wuts, Protective Groups in Organic Synthesis, second edition, New York: John Wiley and Sons (1991), incorporated herein by reference in its entirety). Particularly useful protecting groups include t-butyl esters and ethers, acetals, trityl ethers and amines, acetyl esters, trimethylsilyl ethers, trichloroethyl ethers and esters and carbamates.

The type of building block is not generally limited, so long as the building block is compatible with one more reactive groups capable of forming a covalent bond with other building blocks. Suitable building blocks include but are not limited to, a peptide, a saccharide, a glycolipid, a lipid, a proteoglycan, a glycopeptide, a sulfonamide, a nucleoprotein, a urea, a carbamate, a vinylogous polypeptide, an amide, a vinylogous sulfonamide peptide, an ester, a saccharide, a carbonate, a peptidylphosphonate, an azatides, a peptoid (oligo N-substituted glycine), an ether, an ethoxyformacetal oligomer, thioether, an ethylene, an ethylene glycol, disulfide, an arylene sulfide, a nucleotide, a morpholino, an imine, a pyrrolinone, an ethyleneimine, an acetate, a styrene, an acetylene, a vinyl, a phospholipid, a siloxane, an isocyanide, a isocyanate, and a methacrylate. In certain embodiments, the (B₁)_{M} or (B₂)_{K} of formula (I) each independently represents a polymer of these building blocks having M or K units, respectively, including a polypeptide, a polysaccharide, a polyglycolipid, a polylipid, a polyproteoglycan, a polyglycopeptide, a polysulfonamide, a polynucleoprotein, a polyurea, a polycarbamate, a polyvinylogous polypeptide, a polyamide, a poly vinylogous sulfonamide peptide, a polyester, a polysaccharide, a polycarbonate, a polypeptidylphosphonate, a polyazatides, a polypeptoid (oligo N-substituted glycine), a polyethers, a polythoxyformacetal oligomer, a polythioether, a polyethylene, a polyethylene glycol, a polydisulfide, a polyarylene sulfide, a polynucleotide, a polymorpholino, a polyimine, a polypyrrolinone, a polyethyleneimine, a polyacetates, a polystyrene, a polyacetylene, a polyvinyl, a polyphospholipids, a polysiloxane, a polyisocyanide, a polyisocyanate, and a polymethacrylate. In certain embodiments of the molecule for formula (I), from about 50 to about 100, including from about 60 to about 95, and including from about 70 to about 90% of the building blocks have a molecular weight of from about 30 to about 500 Daltons, including from about 40 to about 350 Daltons, including from about 50 to about 200 Daltons.

It is understood that building blocks having two reactive groups would form a linear oligomeric or polymeric structure, or a linear non-polymeric molecule, containing each building block as a unit. It is also understood that building blocks having three or more reactive groups could form molecules with branches at each building block having three or more reactive groups.

In certain embodiments of the molecule for formula (I), L₁ and L₂ each independently represent a linker. The term “linker molecule” refers to a molecule having two or more reactive groups that is capable of reacting to form a linker. The term “linker” refers to a portion of a molecule that operatively links or covalently bonds G or a hairpin structure to a building block. The term “operatively linked” means that two or more chemical structures are attached or covalently bonded together in such a way as to remain attached throughout the various manipulations the multifunctional molecules are expected to undergo, including PCR amplification.

In certain embodiments of the molecule for formula (I), L₁ is a linker that operatively links B₁ to G. In certain embodiments of the molecule for formula (I), L₂ is a linker that operatively links B₂ to G. In certain embodiments, L₁ and L₂ are each independently bifunctional molecules linking B₁ to G by, in no particular order, reacting one of the reactive functional groups of L₁ to a reactive group of B₁ and the other reactive functional group of L₁ to a reactive functional group of G, and linking L₂ to G by, in no particular order, reacting one of the reactive functional groups of L₂ to a reactive group of B₂ and the other reactive functional group of L₂ to a reactive functional group of G. In certain embodiments of the molecule for formula (I), L₁ and L₂ are each independently linkers formed from reacting the chemical reactive groups of B₁ and G or B₂ and G with commercially available linker molecules including, PEG (e.g., azido-PEG-NHS, or azido-PEG-amine, or di-azido-PEG), or an alkane acid chain moiety (e.g., 5-azidopentanoic acid, (S)-2-(azidomethyl)-1-Boc-pyrrolidine, 4-azidoaniline, or 4-azido-butan-1-oic acid N-hydroxysuccinimide ester); thiol-reactive linkers, such as those being PEG (e.g., SM(PEG)n NHS-PEG-maleimide), alkane chains (e.g., 3-(pyridin-2-yldisulfanyl)-propionic acid-Osu or sulfosuccinimidyl 6-(3′-[2-pyridyldithiol]-propionamido)hexanoate)); and amidites for oligonucleotide synthesis, such as amino modifiers (e.g., 6-(trifluoroacetylamino)-hexyl-(2-cyanoethyl)-(N,N-diisopropyl)-phosphoramidite), thiol modifiers (e.g., 5-trityl-6-mercaptohexyl-1-[(2-cyanoethyl)-(N,N-diisopropyl)]-phosphoramidite, or chemically co-reactive pair modifiers (e.g., 6-hexyn-1-yl-(2-cyanoethyl)-(N,N-diisopropyl)-phosphoramidite, 3-dimethoxytrityloxy-2-(3-(3-propargyloxypropanamido)propanamido)propyl-1-O-succinoyl, long chain alkylamino CPG, or 4-azido-butan-1-oic acid N-hydroxysuccinimide ester)); and compatible combinations thereof.

In certain embodiments, the multifunctional molecule is a molecule of formula (I-A:

[(B₁)_{M}-L₁]_{O}-G, (I-A)

wherein B₁, M, L₁, O, and G, are as defined above for formula (I).

In certain embodiments of the molecule of formula (I-A), from about 10% to 100% of the positional building blocks B₁ at position M, based on the total number of positional building blocks, correlate to a combination of from 2, 3, 4, or 5 coding regions, including from about 20% to 100%, including from about 25% to 100%, including from about 30% to 100%, including from about 35% to 100%, including from about 40% to 100%, including from about 45% to 100%, including from about 50% to 100%, including from about 55% to 100%, including from about 60% to 100%, including from about 65% to 100%, including from about 70% to 100%, including from about 75% to 100%, including from about 80% to 100%, including from about 90% to 100%. Conversely, in certain embodiments of the molecule of formula (I-A), from 1 to about 0 to about 90% of the positional building blocks B₁ at position M correlate to or are identified by a single coding region, including from 0 to about 10%, including from 10 to about 15%, including from 10 to about 20%, including from 10 to about 20%, including from 10 to about 25%, including from 10 to about 30%, including from 10 to about 35%, including from 10 to about 40%, including from 10 to about 45%, including from 10 to about 50%, including from 10 to about 55%, including from 10 to about 60%, including from 10 to about 65%, including from 10 to about 70%, including from 10 to about 80%, including from 10 to about 85%, including from 10 to about 90%.

In certain embodiments, the multifunctional molecule is a molecule of formula (I-B:

[(B₁)_{M}-L₁]_{O}-G-L₂, (I-B)

wherein B₁, M, L₁, O, G, and L₂, are as defined above for formula (I).

In certain embodiments of the molecule of formula (I-B), from about 10% to 100% of the positional building blocks B₁ at position M, based on the total number of positional building blocks, correlate to a combination of from 2, 3, 4, or 5 coding regions, including from about 20% to 100%, including from about 25% to 100%, including from about 30% to 100%, including from about 35% to 100%, including from about 40% to 100%, including from about 45% to 100%, including from about 50% to 100%, including from about 55% to 100%, including from about 60% to 100%, including from about 65% to 100%, including from about 70% to 100%, including from about 75% to 100%, including from about 80% to 100%, including from about 90% to 100%. Conversely, in certain embodiments of the molecule of formula (I-B), from 10 to about 90% of the positional building blocks B₁ at position M correlate to or are identified by a single coding region, including from 10 to about 10%, including from 10 to about 15%, including from 10 to about 20%, including from 10 to about 20%, including from 10 to about 25%, including from 10 to about 30%, including from 10 to about 35%, including from 10 to about 40%, including from 10 to about 45%, including from 10 to about 50%, including from 10 to about 55%, including from 10 to about 60%, including from 10 to about 65%, including from 10 to about 70%, including from 10 to about 80%, including from 10 to about 85%, including from 10 to about 90%.

In certain embodiments, the multifunctional molecule is a molecule of formula (I-C:

G-[(L₂-(B₂)_{K}]_{P}, (I-C)

wherein G, L₂, B₂, K, and P, are as defined above for formula (I).

In certain embodiments of the molecule of formula (I-C), from about 10% to 100% of the positional building blocks B₂ at position K, based on the total number of positional building blocks, correlate to a combination of from 2, 3, 4, or 5 coding regions, including from about 20% to 100%, including from about 25% to 100%, including from about 30% to 100%, including from about 35% to 100%, including from about 40% to 100%, including from about 45% to 100%, including from about 50% to 100%, including from about 55% to 100%, including from about 60% to 100%, including from about 65% to 100%, including from about 70% to 100%, including from about 75% to 100%, including from about 80% to 100%, including from about 90% to 100%. Conversely, in certain embodiments of the molecule of formula (I-C), from 10 to about 90% of the positional building blocks B₂ at position K, based on the total number of positional building blocks, correlate to or are identified by a single coding region, including from 10 to about 10%, including from 10 to about 15%, including from 10 to about 20%, including from 10 to about 20%, including from 10 to about 25%, including from 10 to about 30%, including from 10 to about 35%, including from 10 to about 40%, including from 10 to about 45%, including from 10 to about 50%, including from 10 to about 55%, including from 10 to about 60%, including from 10 to about 65%, including from 10 to about 70%, including from 10 to about 80%, including from 10 to about 85%, including from 10 to about 90%.

In certain embodiments, the multifunctional molecule is a molecule of formula (I-D:

L₁-G-[(L₂-(B₂)_{K}]_{P}, (I-D)

wherein G, L₂, B₂, K, P, and L₁, are as defined above for formula (I).

In certain embodiments of the molecule of formula (I-D), from about 10% to 100% of the positional building blocks B₂ at position K, based on the total number of positional building blocks, correlate to a combination of from 2, 3, 4, or 5 coding regions, including from about 20% to 100%, including from about 25% to 100%, including from about 30% to 100%, including from about 35% to 100%, including from about 40% to 100%, including from about 45% to 100%, including from about 50% to 100%, including from about 55% to 100%, including from about 60% to 100%, including from about 65% to 100%, including from about 70% to 100%, including from about 75% to 100%, including from about 80% to 100%, including from about 90% to 100%. Conversely, in certain embodiments of the molecule of formula (I-D), from 10 to about 90% of the positional building blocks B₂ at position K, based on the total number of positional building blocks, correlate to or are identified by a single coding region, including from 10 to about 10%, including from 10 to about 15%, including from 10 to about 20%, including from 10 to about 20%, including from 10 to about 25%, including from 10 to about 30%, including from 10 to about 35%, including from 10 to about 40%, including from 10 to about 45%, including from 10 to about 50%, including from 10 to about 55%, including from 10 to about 60%, including from 10 to about 65%, including from 10 to about 70%, including from 10 to about 80%, including from 10 to about 85%, including from 10 to about 90%.

According to some embodiments, the molecule of formula (I) can be adapted for polydisplay of multiple encoded portions on one or more ends of G. In certain embodiments, G includes at least one hairpin structure and formula (I:

([(B₁)_{M}-L₁]_{Y})_{O}-G-([L₂-(B₂)_{K}]_{W})_{P} (III)

wherein B₁, M, L₁, O, L₂, B₂, and P are as defined above for formula (I),

G includes an oligonucleotide, the oligonucleotide comprising at least two coding regions, wherein the at least two coding regions are single stranded, and wherein G includes at least one hairpin structure;

Y is an integer from 1 to 5; and

W is an integer from 1 to 5.

In certain embodiments of the molecule of formula (III), from about 10% to 100% of the positional building blocks B₁ at position M or positional building blocks B₂ at position K, based on the total number of positional building blocks, correlate to a combination of from 2, 3, 4, or 5 coding regions, including from about 20% to 100%, including from about 25% to 100%, including from about 30% to 100%, including from about 35% to 100%, including from about 40% to 100%, including from about 45% to 100%, including from about 50% to 100%, including from about 55% to 100%, including from about 60% to 100%, including from about 65% to 100%, including from about 70% to 100%, including from about 75% to 100%, including from about 80% to 100%, including from about 90% to 100%. Conversely, in certain embodiments of the molecule of formula (III), from 10 to about 90% of the positional building blocks B₁ at position M or positional building blocks B₂ at position K, based on the total number of positional building blocks, correlate to or are identified by a single coding region, including from 10 to about 10%, including from 10 to about 15%, including from 10 to about 20%, including from 10 to about 20%, including from 10 to about 25%, including from 10 to about 30%, including from 10 to about 35%, including from 10 to about 40%, including from 10 to about 45%, including from 10 to about 50%, including from 10 to about 55%, including from 10 to about 60%, including from 10 to about 65%, including from 10 to about 70%, including from 10 to about 80%, including from 10 to about 85%, including from 10 to about 90%.

A molecule of formula (IV) is also disclosed,

([(B₁)_{M}-D-L₁]_{Y}-H₁)_{O}-G′-(H₂-[L₂-E-(B₂)_{K}]_{W})_{P} (IV)

wherein,

G includes an oligonucleotide, the oligonucleotide comprising at least two coding regions and at least one terminal coding region, wherein the at least two coding regions are single stranded and the at least one terminal coding region is single or double stranded;

H₁ is a hairpin structure comprising oligonucleotides, wherein H₁ terminates in a 5′ end and is attached to an end of the oligonucleotide G;

H₂ is a hairpin structure comprising oligonucleotides, wherein H₂ terminates in a 3′ end and is attached to an end of the oligonucleotide G;

D is a first building block;

E is a second building block, wherein D and E are the same or different;

B₁ is a positional building block and M represents an integer from 1 to 20;

B₂ is a positional building block and K represents an integer from 1 to 20, wherein B₁ and B₂ are the same or different, wherein M and K are the same or different;

L₁ is a linker that operatively links H₁ to D;

L₂ is a linker that operatively links H₂ to E;

O is an integer from zero to 1;

P is an integer from zero to 1;

provided that at least one of O and P is 1;

Y is an integer from 1 to 5;

W is an integer from 1 to 5; and

wherein each positional building block B₁ at position M and/or B₂ at position K is identified by from 1 to 5 coding regions,

from about 10% to 100% of the positional building blocks B₁ at position M and/or B₂ at position K, based on a total number of positional building blocks, are identified by a combination of from 2 to 5 independent coding regions, and

wherein at least one of the first building block D and second building block E is identified by the at least one terminal coding region.

For formula (III), unless otherwise noted, B₁, M, L₁, O, L₂, B₂, K, and P are as described above for formula (I).

In certain embodiments of formula (IV), the oligonucleotide G′ contains at least one terminal coding region, wherein one or two of the terminal coding regions are single stranded. In certain embodiments, the oligonucleotide G′ contains at least one terminal coding region, wherein one or two of the terminal coding regions are double stranded.

In certain embodiments of the molecule of formula (IV), the oligonucleotide contains at least one, including from one to two, terminal coding regions. In certain embodiments, a terminal coding region is a sequence of nucleotides that is not directly bound to a hairpin structure and terminates in a 5′ end or a 3′ end. In certain embodiments, a terminal coding region is a sequence of nucleotides that is directly bound to a hairpin structure. It is understood that the oligonucleotide will have a 5′ and 3′ direction based on the underlying orientation of the nucleotides, even if both ends of the oligonucleotide are bound by hairpin structures.

In certain embodiments of the molecule of formula (IV), one purpose of the terminal coding region is to facilitate selective hybridization of a hairpin structure containing a complementary sequence to an end of the oligonucleotide during the synthesis of the molecule of formula (IV). In certain embodiments, the terminal coding region contains from about 6 to about 50 nucleotides, including from about 12 to about 40 nucleotides, and including from about 8 to about 30 nucleotides. In certain embodiments, if the terminal coding region contains less than about 6 nucleotides, then the number of available, non-cross-reactive sequences would be too low, which would interfere with accurate encoding of the encoded portion of the molecule of formula (IV). In certain embodiments, if the terminal coding region contains more than about 50 nucleotides then the terminal coding region could become cross reactive and lose too much specificity to selectively hybridize to only one hairpin structure. Such cross reactivity would interfere with the ability of the coding regions to accurately code for the addition of the first building block D and/or the second building block E. In certain embodiments of the molecule of formula (IV), the terminal coding region is single or double stranded.

In certain embodiments of the molecule of formula (IV), H₁ and H₂ are each independently hairpin structures. The term “hairpin structure” as used in the present disclosure refers to a molecular structure that contains from 60% to 100% nucleotides by mass percent, and can hybridize to a terminal coding region of the oligonucleotide G′. In certain embodiments of the hairpin structure, the hairpin structure forms a single, continuous polymer chain, and contains at least one overlapping portion (commonly called a “stem”), wherein the overlapping portion contains a sequence of nucleotides that is hybridized to a complementary sequence of the same hairpin structure. In certain embodiments of the hairpin structure, a bridge structure connects two separate oligonucleotide strands; said bridge structure may be comprised of a polyethylene glycol (PEG) polymer of between 2 and 20 PEG units, including between 3 and 15 PEG units, including between 6 and 12 PEG units. In certain embodiments of the hairpin structure, the bridge structure may be comprised of an alkane chain of up to 30 carbons, or a polyglycine chain of up to 20 units, or comprised of some other chain that bears a reactive functional group. In certain embodiments of the molecule of formula (I), an overlapping portion of H₁ and/or H₂ is bound or attached to a terminal coding region of the oligonucleotide G′. In certain embodiments, H₁ and H₂ each independently contain one, two, three, or four loops.

In certain embodiments of the molecule of formula (IV), H₁ and H₂ each independently include from about 20 to about 90 nucleotides, including from about 32 to about 80 nucleotides, including from about 45 to about 80 nucleotides. In certain embodiments, H₁ and H₂ each independently contains 1, 2, 3, 4, 5, 6, 7, 8, 9, or 10, including from 1 to 5, including from 2 to 4, including from 2 to 3, nucleotides modified with suitable functional groups for facilitating reaction with a linker molecule, or in some cases with a building block, including cases where H₁ and H₂ each independently have been synthesized using bases like, but not limited to, 5′-Dimethoxytrityl-5-ethynyl-2′-deoxyUridine, 3′-[(2-cyanoethyl)-(N,N-diisopropyl)]-phosphoramidite (also called 5-Ethynyl-dU-CE Phosphoramidite, purchased form Glen Research, Sterling Va.). In certain embodiments, H₁ and H₂ each independently include non-nucleotides that have suitable functional groups for facilitating reaction with a linker molecule, or in some cases with a building block, including but not limited to 3-Dimethoxytrityloxy-2-(3-(5-hexynamido)propanamido)propyl-1-O-[(2-cyanoethyl)-(N,N-diisopropyl)]-phosphoramidite (also called Alkyne-Modifier Serinol Phosphoramidite, from Glen Research, Sterling Va.), and abasic-alkyne CEP (from IBA GmbH, Goettingen, Germany). In certain embodiments, H₁ and H₂ each independently include nucleotides with modified bases already bearing a linker, for example H₁ and H₂ each independently could be synthesized using bases like, but not limited to, 5′-Dimethoxytrityl-N6-benzoyl-N8-[6-(trifluoroacetylamino)-hex-1-yl]-8-amino-2′-deoxyAdenosine-3′-[(2-cyanoethyl)-(N,N-diisopropyl)]-phosphoramidite (also called amino-modifier C6 dA, purchased from Glen Research, Sterling Va.), 5′-Dimethoxytrityl-N2-[6-(trifluoroacetylamino)-hex-1-yl]-2′-deoxyGuanosine-3′-[(2-cyanoethyl)-(N,N-diisopropyl)]-phosphoramidite (also called amino-modifier C6 dG, purchased from Glen Research, Sterling, Va.), 5′-Dimethoxytrityl-5-[3-methyl-acrylatel]-2′-deoxyUridine,3′-[(2-cyanoethyl)-(N,N-diisopropyl)]-phosphoramidite (also called Carboxy dT, purchased from Glen Research, Sterling Va.), 5′-Dimethoxytrityl-5-[N-((9-fluorenylmethoxycarbonyl)-aminohexyl)-3-acrylimido]-2′-deoxyUridine,3′-[(2-cyanoethyl)-(N,N-diisopropyl)]-phosphoramidite (also called Fmoc-amino modifier C6 dT, Glen Research, Sterling, Va.), 5′-Dimethoxytrityl-5-(octa-1,7-diynyl)-2′-deoxyuridine, 3′-[(2-cyanoethyl)-(N,N-diisopropyl)]-phosphoramidite (also called C8 alkyne dT, Glen Research, Sterling Va.), 5′-(4,4′-Dimethoxytrityl)-5-[N-(6-(3-benzoylthiopropanoyl)-aminohexyl)-3-acrylamido]-2′deoxyuridine, 3′-[(2-cyanoethyl)-(N,N-diisopropyl)]-phosphoramidite (also called S-B₂-Thiol-Modifier C6-dT, Glen Research, Sterling Va.), and 5-carboxy dC CEP (from IBA GmbH, Goettingen, Germany), N4-TriGl-Amino 2′deoxycytidine (from IBA GmbH, Goettingen, Germany). Suitable functional groups for modified nucleotides and non-nucleotides in H₁ and H₂ include but are not limited to a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a chloroacetate, an aryl halide, a halide, a boronic acid, an alkyne, an azide, and an alkene.

In certain embodiments, one or more of the hairpin structures H₁ and H₂ can be modified with a label, such as a fluorescent label or a radioactive label. Such labels can facilitate the visualization or quantification of molecules for formula (IV). In certain embodiments, one or more of the hairpin structures H₁ and H₂ are modified with a functional group or tether which facilitates processing.

In certain embodiments of the molecule of formula (IV), a benefit of the hairpin structure of H₁ and H₂ is that one or both can allow for the polydisplay of multiple encoded portions at one or both ends of the molecule of formula (IV). Without wishing to be bound by theory, it is believed that the polydisplay of multiple encoded portions at one or both ends of a multifunctional molecule of the present disclosures provides improved selection characteristics under certain conditions.

In certain embodiments of the molecule of formula (IV), D is a first building block. In certain embodiments, when D is present, D is coded for or selected by a terminal coding region of G′ that is directly attached to H₁. In certain embodiments, the terminal coding region of G′ located closest to D corresponds to and can be used to identify the first building block D.

In certain embodiments of the molecule of formula (IV), E is a second building block. In certain embodiments, when E is present, E is coded for or selected by a terminal coding region of G that is directly attached to H₂. In certain embodiments, the terminal coding region of G′ located closest to E corresponds to and can be used to identify the first building block E. In certain embodiments, the first building block D and the second building block E can be the same or different. It is understood that the first building block and second building block are both “building blocks” as described above for formula (I).

In certain embodiments of the molecule of formula (IV), from about 10% to 100% of the positional building blocks B₁ at position M and/or B₂ at position K, based on the total number of positional building blocks, correlate to a combination of from 2, 3, 4, or 5 coding regions, including from about 20% to 100%, including from about 30% to 100%, including from about 50% to 100%, including from about 70% to 100%, including from about 90% to 100%. Conversely, in certain embodiments of the molecule of formula (IV), from 0 to about 90% of the positional building blocks B₁ at position M and/or B₂ at position K correlate to or are identified by a single coding region, including from 0 to about 10%, including from 0 to about 20%, including from 0 to about 30%, including from 0 to about 50%, including from 0 to about 70%.

The present disclosure relates to methods of synthesizing multifunctional molecules, including the molecule of formula (I). As depicted in FIGS. 1-4, in certain embodiments of a method of synthesizing a molecule of formula (I), the method uses a series of “sort and react” steps, where a mixture of multifunctional molecules containing different combinations of coding regions are sorted into sub-pools by selective hybridization of one or more coding regions of the multifunctional molecule with an anti-coding oligomer immobilized on a hybridization array. In certain embodiments of the method, a benefit to sorting the multifunctional molecules into sub-pools is that this separation allows for each sub-pool to be reacted with a positional building block B, including B₁ and/or B₂, under separate reaction conditions before the sub-pools of multifunctional molecules are combined or mixed for further chemical processing. In certain embodiments of the method, the sort and react process can be repeated to add a series of positional building blocks. In certain embodiments of the method, a benefit of adding building blocks using a sort and react method is that the identity of each positional building block of the encoded portion of the molecule can be correlated to from 1, 2, 3, 4, or 5 the coding region(s) that were used to selectively separate or sort the multifunctional molecule prior to the addition of a building block.

In certain embodiments, as depicted in FIGS. 1-2, one or more building blocks can be added by separating a multifunctional molecule into sub-pools using a single sorting step, reacting the multifunctional molecule with a building block, and then remixing. In such an embodiment, the one coding region used to sort the multifunctional molecule during synthesis would unquietly identify or correlate to the building block according to its position, because the identity of the coding region used can be correlated to the identity of the reaction used to add the building block, which would include would include the identity of the positional building block added.

In certain embodiments, as depicted in FIGS. 3-4, one or more building blocks can be added by 2, 3, 4, or 5 sorting steps, reacting the multifunctional molecule with a building block, and then remixing. In such an embodiment, the combination or series of coding regions used to sort the multifunctional molecule during synthesis would uniquely identify or correlate to the building block according to its position, because the combination or series of coding regions used can be correlated to the identity of the reaction used to add the building block, which would include would include the identity or structure of the positional building block added.

In certain embodiments, the method of synthesis can be independently switched from a single sorting step (mononomial expression) or a series of sorting steps (multinomial expression), as desired. In certain embodiments of the method, the from about 10% to 100% of the positional building blocks B₁ at position M and/or B₂ at position K are added by a series of from 2, 3, 4, or 5 sorting steps, including from about 20% to 100%, including from about 30% to 100%, including from about 50% to 100%, including from about 70% to 100%, including from about 90% to 100%.

It is understood that the molecules of formula (I) can include one or more coding regions that are identical between or among molecules in a pool, but it is also understood that the vast majority, of the molecules in the pool would have a different combination of coding regions. In certain embodiments of the method, a benefit of a pool of molecules having a different combination of coding regions is that the different combinations can encode for multifunctional molecules having a multitude of different encoded portions.

In certain embodiments, the method includes providing at least one hybridization array. The step of providing a hybridization array is not generally limited, and includes manufacturing the hybridization array using various techniques, or acquiring an array. In certain embodiments of the method, a hybridization array includes a substrate of at least two separate areas having immobilized anti-codon oligomers on their surface. In certain embodiments, each area of the hybridization array contains a different immobilized anti-codon oligomer, wherein the anti-codon oligomer is an oligonucleotide sequence that is capable of hybridizing with one or more coding regions of a molecule of formula (I). In certain embodiments of the method, the hybridization array uses two or more chambers. In certain embodiments of the method, the chambers of the hybridization array contain a solid matrix, or particles, such as beads, that have immobilized anti-codon oligomers on the surface of the particles. In certain embodiments of the method, a benefit of immobilizing a molecule of formula (I) on the array, is that this step allows the molecules to be sorted or selectively separated into sub-pools of molecules on the basis of the particular oligonucleotide sequence of one or more coding regions. In certain embodiments, the separated sub-pools of molecules can then be separately released or removed from the array into reaction chambers for further chemical processing. In certain embodiments, the step of releasing is optional, not generally limited, and can include dehybridizing the molecules by heating, using denaturing agents, or exposing the molecules to buffer of pH ≥12. In certain embodiments, the chambers or areas of the array containing different immobilized oligonucleotides can be positioned to allow the contents of each chamber or area to flow into an array of wells for further chemical processing.

In certain embodiments, the method includes reacting the at least one building block B, including B₁ and/or B₂, with a multifunctional molecule to form a sub-pool of molecules of formula (I), wherein B₁ and/or B₂ is as defined above for formula (I). In certain embodiments, the building block B₁ and/or B₂ can be added to the container before, during, or after the molecule of formula (I). It is understood that the container can contain solvents, and co-reactants under acidic (e.g. pH from 4-7), basic, or neutral conditions, depending on the coupling chemistry that is used to react the building block B₁ and/or B₂ with the multifunctional molecule to form the molecule of formula (I).

A method of identifying probe molecules capable of binding or selecting for a target molecule is disclosed. In certain embodiments, the method includes exposing a target molecule to a pool of multifunctional molecules, such as a molecule of formula (I), to determine if one of the multifunctional molecules is capable of binding the target molecule. In certain embodiments, the term “exposing” includes any manner of bringing the target molecule into contact with a probe molecule, including a molecule of formula (I). In certain embodiments, the probe molecules that do not bind the target molecule are removed by a removal method, including washing the unbound probe molecules away from the target molecule using excess solvent. In certain embodiments, the target molecule is immobilized on a surface. In certain embodiments, the target molecule includes proteins, enzymes, lipids, oligosaccharides, and nucleic acids with tertiary structures.

In certain embodiments of the method, the amplifying step includes using PCR techniques oligonucleotide in G of formula (I). In certain embodiments of the method, the copy sequence contains a copy of the at least two coding regions of formula (I). In certain embodiments, one benefit of amplifying the oligonucleotide in G from the at least one probe molecule includes the ability to detect which encoded portions of a multifunctional molecule are capable of binding a target molecule, even though the multifunctional molecule cannot easily be removed from the target molecule. In certain embodiments, a benefit of amplification is that it allows for libraries of molecules with vast diversity to be generated. This vast diversity comes at the cost of low numbers of any given molecule of formula (I). Amplifying by PCR allows identification of oligonucleotide sequences present in very small numbers by increasing those numbers until an easily detectable number is reached. Then, DNA sequencing and analysis of the copy sequence can identify or be correlated to the encoded portion of the multifunctional molecule of formula (I) that was capable of binding the target.

The construction of hybridization arrays is described below. Briefly, in certain embodiments, a hybridization array is an array of spatially separated features containing solid supports. In certain embodiments, on these supports are covalently tethered ssDNA oligos with sequences complementary to the sequences of the coding region being sorted. In certain embodiments, by flowing a library of molecules of formula (I), bearing a plurality of coding sequences over or through a solid support bearing a given anti-coding sequence, the members of the library having the complementary coding sequence can be specifically immobilized. In certain embodiments, flowing the library over or through an array of solid supports each of which bears a different immobilized anti-coding sequence will sort the library into subpools based on coding sequence. In certain embodiments, each sequence-specific subpool can then be independently reacted with a specific building block (positional building block) to establish a sequence to building block correspondence. In some embodiments, sequence-specific subpools can be further sorted into more sequence-specific subpools. This synthesis will be described in more detail below, and can be performed on the hybridization array, or after the subpools have been eluted in subpools off of the array into a suitable environment, such as separate containers, for reaction.

Establishing a correspondence between a coding sequence and/or a combination of coding sequences to a building block can be accomplished in the same way, the only difference being that a different hybridization array bearing a different set of anti-coding sequences is used as appropriate.

Coding regions in the oligonucleotides G may also encode other information. In certain embodiments, after translation of the library is complete, it may be desirable to sort the library based on index coding region sequences. In certain embodiments, index coding region sequences can encode the intended purpose, or the selection history of its corresponding subpool of the library. For example, libraries for multiple targets can be translated simultaneously together, and then sorted by the index coding region into subpools. Subpools intended for different targets, and/or for selections under different conditions can be thus separated from each other and made ready for use in their respective applications. The selection history of a library member undergoing multiple rounds of selections for various properties can thus be recorded in the index region.

Many kinds of chemistry are available for use in this invention. In theory, any chemical reaction could be used that does not chemically alter DNA. Reactions that are known to be DNA compatible include but are not limited to: Wittig reactions, Heck reactions, Horner-Wadsworth-Emmons reactions, Henry reactions, Suzuki couplings, Sonogashira couplings, Huisgen reactions, reductive aminations, reductive alkylations, peptide bond reactions, peptoid bond forming reactions, acylations, SN2 reactions, SNAr reactions, sulfonylations, ureations, thioureations, carbamoylations, formation of benzimidazoles, imidazolidinones, quinazolinones, isoindolinones, thiazoles, imidazopyridines, diol cleavages to form glyoxals, Diels-Alder reactions, indole-styrene couplings, Michael additions, alkene-alkyne oxidative couplings, aldol reactions, Fmoc-deprotections, trifluoroacetamide deprotections, Alloc-deprotections, Nvoc deprotections and Boc-deprotections. (See, Handbook for DNA-Encoded Chemistry (Goodnow R. A., Jr., Ed.) pp 319-347, 2014 Wiley, New York. March, Advanced Organic Chemistry, fourth edition, New York: John Wiley and Sons (1992), Chapters 10 to 16; Carey and Sundberg, Advanced Organic Chemistry, Part B, Plenum (1990), Chapters 1-11; and Coltman et al., Principles and Applications of Organotransition Metal Chemistry, University Science Books, Mill Valley, Calif. (1987), Chapters 13 to 20; each of which is incorporated herein by reference in its entirety.)

It will be understood that a vast assortment of different combinatorial scaffolds can be incorporated into multifunctional molecules of the present disclosure. Examples of the kinds of general classes of scaffolds include but are not limited to the following: (a) chains of bifunctional building blocks connected end to end, peptides and peptoids are two examples of this kind of scaffold; it will be appreciated that not every bifunctional building block in the chain will have the same pair of functional groups, and that some building blocks may have only one functional group, e.g. terminal building blocks, (b) branching chains of bifunctional building blocks that include some tri-functional building blocks, and may or may not include mono-functional building blocks, (c) molecules comprised of a single polyfunctional building block, and a set of monofunctional building blocks; in one embodiment, such a molecule may have a polyfunctional building block that acts as a central core, to which other mono-functional building blocks are added as diversity elements, (d) molecules comprised of two or more polyfunctional building blocks to which are connected a set of monofunctional or bifunctional building blocks as diversity elements, (e) any of the above scaffolds that includes formation of a ring by reacting a moiety on the linker or a building block installed at an earlier step with a moiety on a building block or the linker installed at a later step. Other scaffolds or chemical structural phyla can also be incorporated, and these general structural scaffolds are only limited by the ingenuity of the practitioner in designing the chemical pathways to synthesize them.

In certain embodiments, ion-exchange chromatography facilitates the chemical reactions performed on substrates tethered to DNA in two ways. For reactions conducted in aqueous solvent, purification can be readily accomplished by pouring the reaction over an ion exchange resin like DEAE-SEPHAROSE®, or TOYOPEARL® SuperQ 650M. In certain embodiments, the DNA will be bound to the resin by ion exchange, and unused reactants, by-products and other reaction components can be washed away with aqueous buffers, organic solvents or mixtures of both. For reactions that work best in organic solvent, a real problem exists: DNA has very poor solubility in organic solvents, and such reactions suffer from low yields. In these cases, library DNA can be immobilized on ion exchange resin, residual water washed away by a water miscible organic solvent, and the reaction performed in an organic solvent that may or may not be water miscible. See, for example, R. M. Franzini, et. al. Bioconjugate Chemistry 2014 25 (8), 1453-1461, and references therein. Many types and kinds of ion exchange media exist, all having differing properties that may be more or less suited to different chemistries or applications, and which are commercially available from numerous companies like THERMOFISHER®, SIGMA ALDRICH®, DOW®, DIAION® and TOYOPEARL® to name only a few. It will be appreciated that there are many possible approaches and media by which library DNA might be immobilized or solubilized for the purpose of conducting a chemical reaction to install a building block, or remove a protecting group, or activate a moiety for further modification, that are not listed here.

In certain embodiments, a hybridization array comprises a device for sorting a heterogeneous mixture of ssDNA sequences by sequence specific hybridization of those sequences to complementary oligos that are immobilized in a position-addressable format. See, for example, U.S. Pat. No. 5,759,779. It will be appreciated that hybridization arrays may take on many physical forms. In certain embodiments, hybridization arrays possess the ability for a heterogenous sample or ssDNAs (ie. a library of compounds of formula (I)) to come into contact with complementary oligos that have been immobilized on a surface of the array. The complementary oligos will be immobilized on a surface of the array in a manner that enables, allows or facilitates sequence-specific hybridization of the ssDNA to the immobilized oligo, thereby immobilizing the ssDNA as well. In certain embodiments, ssDNAs that have been immobilized through a common sequence can be independently removed from the array to form a subpool.

In some embodiments, the hybridization array will be a chassis comprising a rectangular sheet of plastic between 0.1 and 100 mm thick into which has been cut a series of holes, termed ‘features’. In certain embodiments, on the underside and top of the sheet will be adhered filter membranes. In certain embodiments, in the features, trapped between the filter membranes, will be a solid surface or collection of solid surfaces, termed ‘solid support.’ In certain embodiments, a single sequence of oligo will be immobilized on the solid support in any given feature.

In certain embodiments, a library of molecules of formula (I) can be sorted on the array by allowing an aqueous solution of the library to flow over and through the features. In certain embodiments, as members of the library come in contact with oligos in features bearing complementary sequences, they become immobilized within the feature. In certain embodiments, after hybridization is complete, the features of the array can be positioned over a receiver vessel, like a 96-well plate or a 384-well plate. In certain embodiments, addition of an alkaline solution that causes the de-hybridization of DNA can be added to each feature and the solution will carry the library, now mobile, into the receiver vessel. Other methods of de-hybridizing are also possible, like the use of hot buffer, or denaturing agents. Thus, in certain embodiments, a library of molecules can be sorted into subpools in a sequence specific manner.

It will be appreciated, that the chassis described above could be comprised of plastic, ceramic, glass, polymer or metal. It will be appreciated that the solid supports can be comprised of a resin, glass, metal, plastic, polymer or ceramic, and that the supports can be porous or non-porous. It will be appreciated that higher surface areas on the solid supports allow for larger amounts of complementary oligos to be immobilized and larger amounts of library subpools can be captured in the feature. It will be appreciated that the solid supports can be held in their respective features by filter membranes made of nylon, plastic, cloth, polymer, glass, ceramic or metal. It will be appreciated that the solid supports can be held within their respective features by approaches other than filter membranes, like glue, adhesives, or covalent bonding of the support to the chassis and/or to other supports. It will be appreciated that the features may or may not be holes in a chassis, but independent constructs which can be taken out of or placed in a chassis. It will be appreciated that the shape of the chassis need not be rectangular with features arranged in 2 dimensions, but could be cylinder or rectangular prism with features arranged in one dimension or 3 dimensions. See, for example, U.S. Pat. No. 5,759,779.

Libraries of molecules of formula (I) can be thought of as populations of phenotypes tethered to their respective genotypes. Such a population can be subjected to a selection pressure that removes less fit individuals from the population, and allows more fit members to survive. The oligonucleotide in G genotypes of the second generation population—those surviving selection—can be amplified by PCR, re-translated, and subjected to another, more stringent selection for the same trait, or selected for some orthogonal trait. The subpopulation surviving a selection can also be sequenced, typically using deep sequencing or next-generation sequencing techniques, and the sequencing data can be analyzed to identify the encoded portions (phenotypes) that are the most fit.

Numerous kinds of selection can be performed. The most typical selection is performed to find individuals in the population that are capable of binding to a target protein. In certain embodiments, a method of performing such a selection is to immobilize the target protein on a solid support, like the surface of a well in a NUNC MAXISORP® plate, or by biotinylating the target and immobilizing on streptavidin-coated magnetic beads. In certain embodiments, after immobilization of the target, the population of molecules of formula (I) is incubated with the target on the support. All those individuals capable of binding the target will do so, and become immobilized themselves. Washing the solid support with an appropriate buffer, removes the non-binders. In certain embodiments, the DNA encoding the binders can be amplified by PCR and either sent for sequencing for re-translated and subjected to another round of selection.

In certain embodiments, selections can be performed in a way that selects individuals that bind one target protein to the exclusion of a different, anti-target, protein, or a set of anti-target proteins. In such a case, one method of selection requires both the target and the anti-target(s) be immobilized on solid supports in separate vessels. In certain embodiments, the library is first incubated with the anti-target, and individuals that can bind the anti-target do so. In certain embodiments, the non-binders are carefully removed from the vessel and transferred to the vessel containing the target. In this manner, the population being selected for the ability to bind the target is first depleted in individuals capable of binding the anti-target, and the selection produces individuals whose fitness is characterized as the ability to bind the target or the exclusion of the anti-target.

In certain embodiments, a second method of identifying encoded portions binding one target selectively over another, is to perform parallel selections for both targets, and then eliminate encoded portions demonstrating affinity to both targets during analysis of sequencing data.

In certain embodiments, selections can also be performed that select for binders with long off-rates by using a mixture of immobilized target and free target. In certain embodiments, the library is incubated with immobilized target, allowing binders to bind. Then an excess of free target is added and incubated for a predetermined amount of time. During that time, any binders that release from the immobilized target and then rebind have a high probability of rebinding to the free target. Upon washing away non-binders, the free target, and anything bound to it will also be washed away. The only binders left behind on the free target are those binders whose off-rates are longer than the pre-determined incubation time of the free target.

The methods of selection described in the preceding paragraphs can be found in the literature for phage display, ribosome display, and mRNA display. See, for example, Amstutz, Patrick, et al., Cell biology: a laboratory handbook, 3rd ed. ELSEVIER, Amsterdam (2006): 497-509, and references therein.

In principle, selections can be performed for any property, provided an approach can be constructed that selectively amplifies those individuals in the population that have the property over those individuals that do not. Selections for pharmacologically relevant properties other than target binding are possible in principle and examples include, but are not limited to, selections for water-solubility, cell membrane penetrance, and non-toxicity.

It will also be appreciated, that synthesis of a library in sufficient amounts may allow for more than one selection to be performed in a given round. In certain embodiments, the subpopulation of survivors after a selection for affinity to a target, could be isolated and subjected to a second selection for affinity to the same or to different targets, or selected for an orthogonal property. In some cases, the subpopulation of survivors is purified after selection for affinity to a target and before being subjected to a second selection for affinity. In certain embodiments, the subpool of survivors is then amplified by PCR and sequenced, or it is amplified and re-translated for further selections.

In certain embodiments, the sequencing data is analyzed by comparing the representation of a library member in the population before and after selection. In certain embodiments, members less represented after selection are typically deemed less fit, and those more represented after selection are deemed more fit. In addition, the data is, in some cases, analyzed to determine which individual building blocks confer fitness, which pairs of building blocks confer fitness, and which triplets of building blocks confer fitness when coupled in the same encoded portion. In certain embodiments, the data is analyzed to determine which structural elements within different building blocks and within different encoded portions confer fitness on selected members of the library. In certain embodiments, these analyses inform which members should be synthesized for independent testing, and suggest analogous molecules that should be made and tested which may not be native members of the library. In certain embodiments, three-dimensional docking algorithms can also inform these processes.

In certain embodiments, library members identified in data analysis, can be synthesized with or without the oligonucleotide portion, typically using the same or similar synthetic conditions that were used in making the library. In certain embodiments, these independently synthesized samples can then be subjected to various tests that characterize its physical and chemical properties and suggest its general fitness for a desired task. In certain embodiments, these properties include but are not limited to the dissociation constant or KD which measures the tightness of the library member's binding to its target, its water solubility as measured by a water: octanol partition, and its cell penetrance measured in CaCo cells.

In certain embodiments, identified library members that bind a biomolecule can be used to ascertain the biological function of that biomolecule. In certain embodiments, the functions of many proteins are not known, and the method of the present disclosure provides a ready path to discovery of molecule probes to aid in the elucidation of those functions. In certain embodiments, library members identified by the method of the present disclosure can be used to help determine if a biomolecule is specifically amenable to small molecule discovery and to targeting for drug intervention.

In certain embodiments, the effect on biomolecule function of binding the library member to it can be assayed in in vitro assays or in in vivo assays, in cell-based or in non-cell-based assays. For biomolecules with known function, the effect of the identified library member on that function can be assessed. If the biomolecule is an enzyme, effects on its rates of activity can be assessed. If it is a signaling protein, effects on cellular function can be assessed, including cell viability, cell gene expression, or cellular phenotype expression. If the target is a viral protein, the effect of the library member on viral proliferation and viability can be assessed.

In certain embodiments, library members identified through selections can also be evaluated for their effects on animal and human and plant health in in vivo experiments.

In certain embodiments, library members identified through selections can also be used as affinity reagents for the purification of the biomolecule target. In certain embodiments, the identified encoded portion can be immobilized on a solid support, and a heterogeneous solution containing the target can be flowed over the solid support. In certain embodiments, the target will be bound to the encoded portion, and immobilized. In certain embodiments, all other components of the mixture can be washed away, leaving a purified sample of the target behind.

This invention is illustrated by but not limited by the following examples. Those skilled in the art will recognize many equivalent techniques for accomplishing the steps or portions of the steps enumerated herein.

### EXAMPLES

An embodiment of a molecule of formula (I) is constructed as follows.

### Example 1: Construction of a 16M—Member Gene Library (Molecules of Formula G)

### Example 1a. Design and Provision of Codons for the Gene Library

16 double-stranded DNA (“dsDNA”) sequences are provided or purchased from a gene synthesis company like Genscript in Piscataway N.J., Synbio Technologies in Monmouth Junction N.J., Biomatik of Wilmington Del., Epoch Life Sciences of Sugarland Tex., among others. These sequences comprise 6 coding regions of 20 bases each. Each codon is flanked by a 20-base non-coding region (making a total of 7 non-coding regions). All of the coding region sequences are unique, and chosen to be un-cross-reactive with other coding sequences and with the non-coding regions. The 7 non-coding regions in a DNA molecule have different sequences, but the sequence at each position is conserved across all the DNAs.

Coding and non-coding regions are designed in silico as follows. All coding and non-coding regions are designed to have similar melting temperatures (typically between 58° C. and 62° C.). DNA sequences are generated randomly in silico. Once generated, the sequence melting temperature and thermodynamic properties (delta H, delta S and delta G of melting) are calculated using the nearest neighbor method. If the calculated Tm and other thermodynamic properties are not within the predefined range desired for the library, the sequence is rejected. Acceptable sequences are subjected to analysis by sequence similarity algorithms. Sequences predicted by the algorithm to be sufficiently non-homologous are presumed to be non-cross-reactive, and are kept. Others are rejected. Coding and non-coding regions are sometimes chosen from empirical lists of oligos shown to be non-cross hybridizing. See Giaever G, Chu A, Ni L, Connelly C, Riles L, et al. (2002). Functional profiling of the Saccharomyces cerevisiae genome. Nature 418: 387-391. This reference lists 10,000 non-cross-reactive oligos. The Tm of each is calculated and those falling within the predefined range are analyzed by sequence homology algorithms. Those which are sufficiently non-homologous are retained.

Each non-coding region contains a unique restriction site. The non-coding region at the 5′ end of the template strand contains a SacI recognition site at bases 13-18 from the 5′ end. The non-coding region at the 3′ end of the coding strand contains an EcoRI restriction site at bases 14-19 from the 3′ end of the template strand. The second, third, fourth, fifth and sixth non-coding regions from the 5′ end of the template strand have HindIII, NcoI, BamHI, NsiI, and SphI recognition sites respectively at bases 8-13.

### Example 1b. The DNAs are Restriction Digested to De-Couple all Codons from Each Other

The DNA sequences are pooled and dissolved in CUTSMART® buffer from New England Biolabs (NEB, Massachusetts) at a concentration of about 20 μg/ml. The internal restriction enzymes are added and the digestion is done for 1 hour at 37° C., following the enzyme manufacturer's protocols. The enzymes are heat inactivated at 80° C. for 20 minutes. After inactivation, the reaction is held at 60° C. for 30 minutes, then cooled to 45° C. and held for 30 minutes, and then cooled to 16° C.

### Example 1c. The Codons are Combinatorially Re-Assorted to Produce a Gene Library

To re-assemble the individual coding regions produced in the digestion reaction into full-length genes, T4 DNA Ligase from NEB is added to the reaction to 50 U/ml, dithiothreitol (DTT, Thermo Fisher Scientific, Massachusetts) is added to 10 mM, and adenosine 5′-triphosphate (ATP, from NEB) is added to 1 mM in accordance with the manufacturer's protocol. The ligation reaction is performed for 2 hours, and the product is purified by agarose gel electrophoresis. Because the sticky-ends produced by digestion at one site in a non-coding region of a provided gene will anneal only to the sticky-ends of all the other digestion products at the same site, a complete combinatorial re-assortment will occur. Thus, with 16 coding sequences at each of 6 coding positions, and using a binomial encoding strategy where 2 coding regions are required to encode a given building block, the number of library members that can be encoded is ((16²)³=) 16.8 million members.

### Example 2a: Prepare the Gene Library by an Alternate Method

Example 1 describes the combinatorial re-assortment of all codons simultaneously by restriction digestion at all internal non-coding regions of provided library gene sequences followed by ligation. In some cases, this process is done in a step-wise fashion. The same digestion reaction conditions are used except, a single restriction endonuclease is added, instead of all the endonucleases. Then using the same ligation reaction conditions the restriction digestion products are re-ligated together. The ligation product is purified by agarose gel electrophoresis, amplified by PCR, and then cut by the next restriction enzyme. The process is repeated until the gene library is complete.

### Example 2b: Prepare the Gene Library by a Second Alternate Method

In some embodiments, incomplete combinatorial re-assortment of codons to produce a population with markedly lower complexity would be advantageous. Such a gene library is produced by first splitting a mixture of the 16 gene sequences described in Example 1 into several aliquots. Each aliquot is then restriction digested by a different combination of internal restriction enzymes, using the same reaction conditions. After heat inactivation of the restriction enzymes, the independent digestion products are re-ligated as per the protocol. The products are pooled and purified by agarose gel electrophoresis, amplified by PCR, and the rest of library preparation and translation and selection is done as per Examples following.

### Example 2c. Prepare the Gene Library by a Third Alternate Method

The library is prepared as before with the following exceptions. The library is constructed by purchasing two sets of oligos, a coding strand set of oligos and an anti-coding strand set of oligos. Each set comprises as many subsets as there are coding regions, and as many different sequences are in each subset as there are different coding sequences at a coding region. Each oligo in each subset of the coding strand oligos comprises a coding sequence and, in some cases, a 5′ non-coding region. Each oligo in each subset of the anti-coding strand oligos comprises an anti-coding sequence and, in some cases, a 5′ non-coding region complement. In order to facilitate ligations downstream in the process, all the oligos except those for the 5′ termini of the coding and anti-coding strands are purchased with 5′ phosphorylations, or are phosphorylated with T4 PNK from NEB as per the manufacturer's protocol. The subset of oligos possessing the coding strand 5′ terminal coding sequences is combined in T4 DNA Ligase buffer from NEB with the subset possessing the 3′ terminal anti-coding sequences, and the two sets are allowed to hybridize. Doing so produces a product comprising a single-stranded 5′ overhang non-coding region on the coding strand, a double-stranded coding region, and an optional single stranded 5′ overhang non-coding region on the anti-coding strand. This hybridization procedure is carried out separately for each coding/anti-coding pair of oligo subsets. For example, the subset of sequences encoding the second coding region from the 5′ end is hybridized with its complementary anti-coding subset, the subset encoding the third coding region from the 5′ end with its complementary subset, and so forth. The hybridized subset pairs are pooled and, in some cases, purified by agarose gel electrophoresis. If the genes in the library possess non-coding regions of 1 base or more in length, and if the non-coding regions between coding regions are unique, then equimolar amounts of each hybridized subset pair are added to a single vessel. The single-stranded non-coding regions hybridize, and are ligated to each other by T4 DNA Ligase from NEB using the manufacturer's protocol. If the non-coding regions are 1 base in length or more, but are not unique, then two adjacent hybridized subsets are added to one vessel, the single-stranded non-coding regions anneal, and are ligated with T4 DNA Ligase. Upon reaction completion, the product is, in some cases, purified by agarose gel electrophoresis, and a third hybridized subset that is adjacent to one of the ends of ligated product is added, annealed and ligated. This process is repeated until construction of the library is complete. It will be appreciated that libraries comprised of arbitrary numbers of coding regions are constructed by this method. For current purposes, libraries of more than 20 coding regions may be impractical for reasons unrelated to library construction. It will be appreciated that blunt ligations are commonly performed by those skilled in the art, and that coding regions do ligate without intervening non-coding regions, but that for hybridized subsets possessing no non-coding regions at either end, that the ligation provides both sense and anti-sense products. Products possessing the correct sense are purified away from products possessing anti-sense by preparing the library and sorting it on all hybridization arrays sequentially. The portion of the library that is captured on the array at each hybridization step possesses the correct sense. It will be appreciated that a non-coding region comprised only of a unique restriction site sequence is an attractive option of this method.

### Example 2d. Purchase of a Gene Library

Gene libraries like the one described in Examples 1 and 2 can be purchased from Twist Bioscience of 500 Terry Francois Boulevard, San Francisco, Calif. 94158.

### Example 3: Prepare Translation-Ready, Single-Stranded Oligonucleotide G

### Example 3 a. Amplify the Gene Library by PCR

A T7 promoter is appended to the 5′ end of the non-template strand by extension PCR using these reactants for a 50 μL reaction: 5× PHUSION® High-Fidelity DNA Polymerase (“PHUSION® Polymerase”, NEB), 10 μL; deoxynucleotide (dNTP) solution mix 200 04 final concentration; forward primer, final concentration 750 nM; reverse primer final concentration, 750 nM; template (enough template should be used to adequately oversample the library); dimethyl sulfoxide (DMSO), 2.5 uL; “PHUSION® Polymerase”, 2 μL. Perform the PCR using an annealing temperature of 57° C., and an extension temperature of 72° C. Anneal for 5 seconds each cycle; extend for 5 seconds each cycle. Analyze the product by agarose gel electrophoresis.

### Example 3b. Transcribe the DNA into RNA

Without purification of the PCR product, a 250 μL transcription reaction is done with the following reactants: PCR product, 25 μL; RNAse-free water, 90 μL; nucleoside triphosphate's (NTP), 6 mM final concentration in each; 5×T7 buffer, 50 μL; NEB T7 RNA polymerase 250 units; in some cases, RNasin® Ribonuclease Inhibitors (Promega Corporation, WI) can be added to 200 U/ml; in some cases, pyrophosphatase can be added to 10 μg/ml. 5×T7 buffer contains: 1M HEPES-KOH (4-(2-hydroxyethyl)-1-piperazineethanesulfonic acid) pH 7.5; 150 mM magnesium acetate; 10 mM spermidine; 200 mM DTT. The reaction is conducted at 37° C. for 4 hours. The RNA is purified by lithium chloride precipitation. Dilute the transcription reaction with 1 volume of water. Add LiCl to 3M. Spin at maximum g, at 4° C. for at least 1 hr. Decant the supernatant and keep it. A clean pellet will be a clear, glassy, gel that can be difficult to dissolve. Alternating gentle warming (a minute at 70° C.) and gentle vortexing will cause the pellet to re-suspend. Analyze by agarose gel electrophoresis, quantitate and freeze as soon as possible to avoid degradation. See, for example, Analytical Biochemistry 195, p 207-213. (1991), and Analytical Biochemistry 220, p 420-423, (1994).

### Example 3c. Reverse Transcribe the RNA into DNA

The single stranded RNA (“ssRNA”) is reverse transcribed in a 2 step procedure using SUPERSCRIPT® III Reverse Transcriptase from Thermo Fisher Scientific and the supplied First Strand Buffer. The first step is done with these final concentrations of the following components: dNTP's, 660 04 each; RNA template, ˜5 μM; primer, 5.25 μM. The Step 1 components are heated to 65° C. for 5 minutes, then iced for at least 2 min. The Step 2 components final concentrations are: First Strand Buffer, 1×; DTT, 5 mM; RNase Inhibitor (NEB), 0.01 U/uL, SUPERSCRIPT® III Reverse Transcriptase, 0.2 U/μl. The Step 2 components are combined, warmed to 37° C., and after the Step 1 components have been iced 2 minutes, the Step 2 mix is added to the Step 1 mix. The combined parts are reacted at 37° C. for 12 hours. The reaction is followed by agarose gel electrophoresis. Take samples of the reaction, of known starting material RNA and of known product, or known product analog like PCR product library. Add ethylene-diamine-tetra-acetic acid (“EDTA”) to all samples, heat to 65° C., 2 minutes, flash cool, and then run on an agarose gel. ssRNA should resolve from complementary DNA (“cDNA”) product. The cDNA product is purified by adding 1.5 volumes of isopropanol and ammonium acetate to 2.5 M, followed by centrifugation at 48,000 g for 1 hour. The cDNA pellet is re-suspended in distilled water (“dH₂O”) and the RNA strand is hydrolyzed by adding LiOH to pH 13. The solution is heated to 95° C. for 10 minutes. 1.05 equivalents of primers specific for the non-coding regions are added, the pH is brought to neutral with tris(hydroxymethyl)aminomethane (“Tris”) and acetic acid, and the reaction is allowed to cool to room temperature slowly, whereupon it is concentrated and, in some cases, purified.

### Example 3d: Prepare G with a Linker and Reactive Functional Group at the 5′ End During Reverse Transcription

A reactive chemical functional group can be tethered to the oligonucleotide by following the reverse transcription protocol above except the primer used for the reverse-transcription reaction is provided with linker that is placed at or near the 5′ end of the primer. Appropriate linkers are commercially available and include alkyl chains, peptide chains, polyethylene glycol chains, and they are discussed more fully herein. Appropriate chemical functional groups are commercially available already tethered to linkers and include amines, alkynes, carboxylic acids, thiols, alcohols, and are discussed more fully herein. One example of a linkered functional group that can be purchased as part of an oligonucleotide primer is N4-TriGl-Amino 2′deoxycytidine (from IBA, Goettingen, Germany). Primers as described here can be purchased from DNA oligo synthesis companies like Sigma Aldrich, Integrated DNA Technologies of Coralville, Iowa, or Eurofins MWG of Louisville, Ky.

### Example 4. Prepare Molecules of Formula (I), and Formula (II) by Sorting a Library of Oligonucleotide G into a First Set of Sub-Pools, then Sorting Each Sub-Pool into a Second Set of Sub-Pools and Performing Chemistry Specific to Each Sub-Pool

### Example 4a. Preparation of a Hybridization Array

Hybridization arrays are constructed of a TECAFORM™ (Acetal Copolymer) chassis ˜2 mm thick, with holes cut by a computer numerical control machine. A nylon 40 micron mesh from ELKO FILTERING is adhered to the bottom of the chassis using P905 double-sided tape from Nitto Denko. The holes are then filled with a solid support of CM SEPHAROSE® resin (Sigma Aldrich) which has been functionalized with an azido-group. The resin is functionalized using azido-PEG-amine with 8 PEG units purchased from Broadpharm (San Diego, Calif.). 45 ml of packed CM SEPHAROSE® is loaded into a fitted funnel and washed with DMF. The resin is then suspended in 90 ml of DMF and reacted with 4.5 mM azido-PEG-amine, 75 mM EDC, 7.5 mM HOAt, 12 hours at room temp. The resin is washed with DMF, water, isopropanol and stored in ethanol 20% at 4° C. A nylon 40 micron mesh is then adhered to the top of the chassis. The azido group allows alkyne-linked oligos to be tethered to the solid support using click chemistry. Placing the array in an array-to-well-plate adapter, and stationing the adapter over a well plate enables capture oligos to be ‘clicked’ onto the azido-SEPHAROSE® in register. A 30 μl solution containing 1 nmol of alkynyl oligo, copper sulfate, 625 μM tris(3-hydroxy-propyl-triazolyl-methyl)amine (“THPTA”) (ligand), 3.1 mM amino-guanidine, 12.5 mM ascorbate, 12.5 mM phosphate buffer pH 7, 100 mM, is added to each well of the array-to-well-plate adapter and allowed to adsorb onto the SEPHAROSE® support. After 10 minutes, the solution is spun in a centrifuge out of the array and into the plate, whereupon it is re-pipetted in register back onto the array for a second pass at the reaction. After a second 10 minute reaction, the reaction solutions are spun into the well plate, and the well plate is set aside. The array is washed well with 1 mM EDTA, and stored in phosphate buffer solution (“PBS”) with 0.05% sodium azide. The reaction solutions are each diluted to 100 μl with dH₂O, loaded onto diethylaminoethyl (DEAE) ion exchange resin, washed with dH₂O to remove all reagents and reaction by-products except for any un-incorporated oligo, which is eluted off with 1.5M NaCl+50 mM NaOH. These solutions are analyzed by high-performance liquid chromatography (HPLC) to ascertain the degree of incorporation by disappearance of starting material. One array bears oligos complementary to one coding position in the template library. A separate array is made for each coding position.

In some cases, the capture oligos can be immobilized on solid supports as above but in a series of columns in lieu of an array. Many different solid supports other than CM SEPHAROSE® are usable, including cellulose, non-porous beads bearing hydrophilic coatings, among others.

### Example 4b. Sorting a Library by Sequence-Specific Hybridization at a First Coding Region

The hybridization-ready library is diluted to 13 ml in 1× Hybridization Buffer (2× saline sodium citrate (SSC), +15 mM Tris pH7.4+0.005% TRITON® X100, 0.02% SDS, 0.05% sodium azide). 10 μg of ‘dummy’ DNA bearing orthogonal sequences are added to block non-specific nucleic acid binding sites. An array is chosen corresponding to the desired coding position in the template library. The array is placed in a chamber that provides 1-2 mm of clearance on either side, and the 13 ml library solution is poured in. The chamber is sealed and rocked gently for 48 hours at 37° C. In some cases, the array is placed in a device that allows the solution containing the library to be pumped in a directed fashioned though the various features in a pre-patterned path as an approach to sort the library on the array faster.

### Example 4c. Eluting Sorted Library Off of a Hybridization Array

The array is washed by unsealing the chamber and replacing the hybridization solution with fresh 1× hybridization buffer, followed by rocking at 37° C. for 30 minutes. The wash is repeated 3 times with hybridization buffer, then 2 times with ¼× hybridization buffer. The library is then eluted off of the array. The array is placed in an array-to-well-plate adapter, and 30 μl of 10 mM NaOH, 0.005% TRITON® χ-100 is added to each well and incubated 2 minutes. The solution is spun in a centrifuge through the array into a well plate. The elution procedure is done 3 times. The sorted library solutions are neutralized by adding 9 μl of 1M Tris pH 7.4 and 9 μl of 1M HOAc, in that order, to each well.

### Example 4d. Sorting a Library by Sequence-Specific Hybridization at a Second Coding Region

Each sub-pool generated by the first sorting is then independently sorted into a second set of sub-pools by sorting each of the first sub-pools on arrays complementary to a second coding region. For example, if the first sorting is performed by hybridizing to an array bearing capture oligos that are complementary to the coding region that is closest to the 5′ end of the oligonucleotide, each of those sub-pools can then be independently sorted on arrays bearing capture oligos complementary to the any other pre-determined coding region.

### Example 4e. Performing a Peptoid Coupling Chemical Step on a Sorted Library

15 μl aliquots of SuperQ 650M resin are added to each well of a filter plate, and washed with 100 μl of 10 mM HOAc. The sorted library is transferred into the well plate bearing the ion exchange resin. The resin and library are washed 1×90 μl with 10 mM HOAc, 2×90 μl with dH₂O, 2×90 μl DMF, 1×90 μl piperidine. Separately, make a solution containing 100 mM sodium chloroacetate and 150 mM 4-(4,6-Dimethoxy-1,3,5-triazin-2-yl)-4-methyl morpholinium chloride in methanol. Add 40 μl of this solution to each well of resin and react at room temperature for 30 minutes. Wash the resin 3×90 μl methanol, then repeat the coupling and wash 3×90 μl methanol, 3×90 μl DMSO. Separately, make 2M (or saturated where necessary) solutions of secondary amines in DMSO. Add 40 μl of one secondary amine solution to each well of resin and react at 37° C. for 12 hrs. Wash the resin 3×90 μl DMSO, 3×90 μl 10 mM acetic acid (HOAc), 3×90 μl dH₂O. Elute the DNA library off of the ion exchange resin with 1.5 M NaCl, 50 mM NaOH, 0.005% TRITON® X-100 in 3×30 μl portions. Pool all the reactions, and neutralize the solution by addition of Tris to 15 mM and HOAc to pH 7.4. Concentrate and buffer exchange into 1× hybridization buffer.

### Example 4f. Complete the Synthesis of the Library

Using the protocols above for sorting the library on hybridization arrays, and using the protocols above for performing peptide or peptoid chemistry, or those below in other Examples for performing other chemical steps, more steps of sorting and synthesis are done and the library is fully translated.

### Example 4g. Sorting a Library by Sequence-Specific Hybridization at a First Coding Region

11 nmol of hybridization-ready library is diluted to 22 ml in 1× Hybridization Buffer (2× saline sodium citrate (SSC), +15 mM Tris pH 7.4+0.005% TRITON® X100, 0.02% SDS, 0.05% sodium azide). 10 μg of tRNA is added to block non-specific nucleic acid binding sites. An array bearing 4 different capture sequences corresponding to the 4th coding position from the 5′ end in the template library. The array is placed in a chamber that provides 1-2 mm of clearance on either side, and the 22 ml library solution is poured in. The chamber is sealed and rocked gently for 20 hours at 61° C., then for 2 hours at 56° C., and 1 hour at 52° C. and 1 hour at 42° C. The array is washed by rocking the array in a chamber with 50 ml of 5× Hybridization Buffer at 56° C. for 15 minutes, followed by rocking in 0.2× Hybridization Buffer for 15 minutes at 38° C. The array was then placed in an adapter device and washed by adding 70 ul of 0.2× Hybridization Buffer to each well and spinning the wash buffer through the features into a receiver plate. The hybridized DNA was eluted by adding 30 ul of Array Elute Buffer (10 mM KOH+0.02% SDS) and spinning the buffer through the array into 384-well UV Clear plate. This elution was done 3 times. The yield of library on the 1st, 2nd, 3rd and 4th sequences on the array were respectively: 1.6 nmol, 1.2 nmol, 2.0 nmol, and 4.8 nmol. Sorting using this procedure has produced subpools with >90% fidelity. This procedure can be repeated until sufficient amount of library has been sorted for next steps.

### Example 4h. Sorting a Library by Sequence-Specific Hybridization at a Second Coding Region

11 nmol of hybridization-ready library is diluted to 22 ml in 1× Hybridization Buffer (2× saline sodium citrate (SSC), +15 mM Tris pH 7.4+0.005% TRITON® X100, 0.02% SDS, 0.05% sodium azide)+700 mM NaCl, bringing the total concentration of NaCl in buffer to 1M. 10 μg of tRNA is added to block non-specific nucleic acid binding sites. An array bearing 96 different capture sequences corresponding to the 5th coding position from the 5′ end in the template library. The array is placed in a chamber that provides 1-2 mm of clearance on either side, and the 22 ml library solution is poured in. The chamber is sealed and rocked gently for 20 hours at 61° C., then for 2 hours at 56° C., and 1 hour at 52° C. and 1 hour at 42° C. The array is washed by rocking the array in a chamber with 50 ml of 5× Hybridization Buffer at 56° C. for 15 minutes, followed by rocking in 0.2× Hybridization Buffer for 15 minutes at 38° C. The array was then placed in an adapter device and washed by adding 70 ul of 0.2× Hybridization Buffer to each well and spinning the wash buffer through the features into a receiver plate. The hybridized DNA was eluted by adding 30 ul of Array Elute Buffer (10 mM KOH+0.02% SDS) and spinning the buffer through the array into 384-well UV Clear plate. This elution was done 3 times. The total yield of library was 6.6 nmol. The average amount of library captured bearing the 96 different sequences was 68 pmol, the maximum amount captured with a sequence was 90 pmol, and the minimum captured with a sequence was 12 pmol.

### Example 4i. Demonstration of Multinomial Encoding, and Use of Library Lacking a Double-Stranded Non-Coding Region

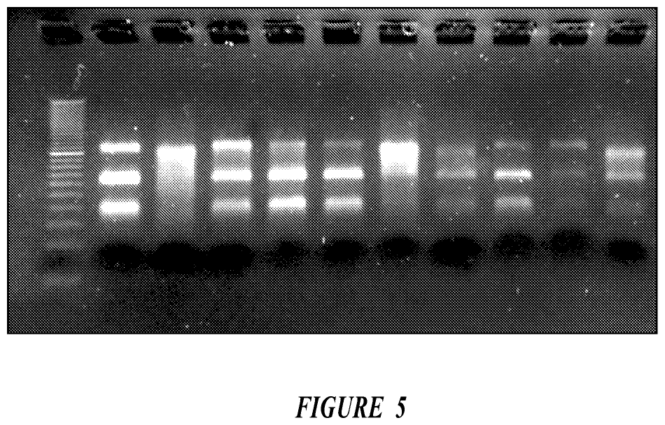

Two library members were prepared, an experimental and a control library member. The experimental oligo possessed 2 adjacent coding regions cognate to 2 capture oligos immobilized on 2 different samples of SEPHAROSE® resin, but this experimental library member did not possess a double-stranded non-coding region between the two adjacent coding regions. Instead it possessed the single-stranded sequence AAATTT. The control library member possessed 2 coding regions that were non-cognate to either of the 2 capture oligos on the resins, it also possessed a double-stranded non-coding region that bore an NcoI restriction site between its non-cognate coding regions. A significant excess of the control library member was added to the experimental library member and they were mixed and allowed to hybridize to the first resin. The resin was drained, washed, and eluted, and the flow-through, wash, and elution were collected separately. The eluted material was mixed with a second significant excess of the control library member, and this sample was hybridized to the second capture oligo on the second resin. The resin was drained, washed, and eluted. Flow through, washes, and elution were collected separately. All the collected samples were subjected to restriction digestion with NcoI. Referring to FIG. 5, the initial mixture of library members showed 3 bands, consistent with an incomplete digestion of the control library member into 2 fragments (and a parent), the experimental library member was too faint to directly observe by gel. The first flow through and first wash also showed the same 3-band pattern, but the wash sample contained enough of the experimental library member that it begins to be visible on the gel. The first elution showed only one strong band, consistent with the presence of only the undigested experimental library member in the sample. This indicates the hybridization was specific for the first coding region possessed by the experimental oligo. The second mixture comprising the first eluted library member and a second aliquot of the control library member showed the 3-band pattern upon restriction digestion, as did the second flow through, and the second wash. This is consistent with the presence of both the experimental and control library members. The second elution showed a significant preponderance of the experimental library member over the control, consistent with enrichment of the experimental library member through specific hybridization on the second resin.

Referring to FIG. 5,

Lane 1: Ladder

Lane 2: Control Library Member Parent and 2 fragment bands from digestion with NcoI

Lane 3: Experimental Library member undigested by NcoI (Note: the Experimental

Library member is about 14 bases shorter than the Control and therefore resolves from it)

Lane 4: First Mix of Experimental and Control Library Members before first hybridization

Lane 5: First hybridization flow through

Lane 6: First hybridization wash

Lane 7: First Elution showing vast excess of Experimental Library Member

LAne 8: Second Mix of Experimental and Control Library Members before second hybridization (Note: the complete digestion of Control Library Member in this lane, and the relative concentration of Control and Experimental Library members)

Lane 9: Second hybridization flow through

Lane 10: Second wash

Lane 11: Second elution. (Note the complete digestion of Control Library member in this lane and the significant increase in the relative proportion of Experimental vs Control Library Member)

Lanes 12 and 13 are not in use.

### Example 5. Perform Selections of Encoded Molecules

### Example 5a. Prepare the Library for Selection

In some cases, once translation of the library is complete, the single-stranded regions are made double-stranded by combining the library as template in dH₂O at less than or equal to 1.0 μM, DREAMTAQ™ buffer at 1×, dNTP's at 1000× [template], DREAMTAQ™ Polymerase at 0.2 U/μl, and a supplement of an equimolar amount of MgCl₂ for each dNTP. Note that the oligo complementary to the 3′ terminal non-coding region or a reaction site adapter at the 3′ end will act as the primer for this reaction. Heat the mixture to 95° C. for 2 minutes, then anneal at 57° C. for 10 seconds and extend at 72° C. for 10 minutes. Purify the reaction by ethanol precipitation.

### Example 5b. Select Ligands that Bind to Protein Target of Interest

5 μg of streptavidin in 100 μl of PBS is immobilized in 4 wells of a MAXISORP™ plate with rocking at 4° C. overnight. The wells are washed with PBST 4×340 μl. Two of the wells are blocked with 200 μl of casein, and 2 others with BSA at 5 mg/ml for 2 hours at room temperature. The wells are washed with PBST 4×340 μl. 5 μg of a biotinylated target protein in 100 μl of PBS are added to a well blocked with casein, and to a well blocked with BSA and incubated with rocking at room temperature for 1 hour (for a protocol on the biotinylation of proteins, see Elia, G. 2010. Protein Biotinylation. Current Protocols in Protein Science. 60:3.6:3.6.1-3.6.21). A 100 μl aliquot of the translated library in PBS with Tween 20 (PBST) is added to each of the wells that did NOT receive the target protein, and 100 μl of PBST is added to the two wells that did receive target protein. The samples are incubated with rocking at room temperature for 1 hour. The buffer is carefully aspirated from the wells containing immobilized target protein and PBST only. The buffer containing library in wells without the target is carefully transferred to target-containing wells. 100 μl PBST are added to the wells without target. All are incubated for 4 hours with rocking at room temperature. The library is carefully removed with a pipette and stored. The wells are washed with 4×340 μl PBST. To elute library members binding tightly to the target protein, an excess of biotin in 100 μl of PBST is added to the wells and incubated for 1 hour at 37° C. The buffer is carefully aspirated and used as the template for a PCR reaction. Tight binders can also be elute using a collection buffer at a temperature hot enough to denature the target protein.

### Example 6. Analyze Selection Results

PCR products from the library before and after selection are submitted for deep sequencing using the primers and protocols required by the DNA Sequencing service provider. Providers include Seqmatic of Fremont Calif., and Elim BioPharm, Hayward, Calif. The coding sequences at the terminal and internal coding regions of each sequenced strand are analyzed to deduce the building blocks used in synthesis of the encoded portion. The relative frequency of identified library members before selection and after suggests the degree to which the library member is enriched in the population by the selection. Analysis of the various chemical subgroups comprising the library members surviving selection shows the degree to which those moieties confer fitness on a library member and are used to evolve more fit molecules or to predict analogous molecules for independent synthesis and analysis.

### Example 7: Index Molecules of Formula (I)

A coding region is set aside or added for use as an indexing region. After preparation and translation of a library as per Examples 1-4, the library is sorted on a hybridization array by a coding region set aside for indexing. The sub-pools generated by such sorting are used for different purposes, are selected for different properties, for different targets, or for the same target under different conditions. In some cases, the products of the different selections are amplified by PCR independently, re-pooled with the other sub-pools, and re-translated as in Examples 1-4.

### Example 8: Perform a Gene-Shuffling or Crossing-Over Reaction on a Library

After a library is translated and selected, performing a gene-shuffling will produce new offspring phenotypes not previously extant in the library, or produce offspring phenotypes that re-sample phenotypes surviving selection. The post-selection library is amplified by PCR. The PCR product is split into a number of aliquots, and each aliquot is subjected to the protocol described in Example 2b. In some cases, each aliquot is subjected to the protocol the protocol described in Example 1 where DNAs are restriction digested to de-couple all codons from each other, and the codons are combinatorially re-assorted to produce a gene library. The digestion/re-ligation products are pooled, purified and amplified as described in Example 2, and subsequent rounds of library preparation, translation and selection is done as per Examples above.

### Example 9: Synthesize an Encoded Portion Using Suzuki Coupling Chemistry

A DNA library bearing an aryl-iodide, either as a reactive site on a reaction site adapter, as a building block on a charged reaction site adapter or as a partially translated molecule, is dissolved in water at 1 mM. To it is added 50 equivalents of a boronic acid as a 200 mM stock solution in dimethylacetamide, 300 equivalents of sodium carbonate as a 200 mM aqueous solution, 0.8 equivalents of palladium acetate as a 10 mM stock solution in dimethylacetamide premixed with 20 equivalents of 3,3′,3″ phosphinetriyltris (benzenesulfonic acid) trisodium salt as a 100 mM aqueous solution. The mixture is reacted at 65° C. for 1 hour then purified by ethanol precipitation. The DNA library is dissolved in buffer to 1 mM and 120 equivalents of sodium sulfide as a 400 mM aqueous solution is added, then reacted at 65° C. for 1 hour. The product is diluted to 200 μl with dH₂O and purified by ion exchange chromatography. (See Gouliaev, A. H., Franch, T. P. O., Godskesen, M. A., and Jensen, K. B. (2012) Bi-functional Complexes and methods for making and using such complexes. Patent Application WO 2011/127933 A1.)

### Example 10: Synthesize an Encoded Portion Incorporating an Imidazopyridine

A DNA library bearing an aryl aldehyde, either as a reactive site on a reaction site adapter, as a building block on a charged reaction site adapter or as a partially translated molecule, is dissolved in borate buffer pH 9.4 at 1 mM. To it is added 50 equivalents of a 2-amino pyridine as a 200 mM stock solution in DMA, and 2500 equiv. of NaCN as a 1M aqueous solution and reacted at 90° C. for 10 hours. The product is purified by ethanol precipitation, or ion exchange chromatography. (See (1) Alexander Lee Satz, Jianping Cai, Yi Chen, Robert Goodnow, Felix Gruber, Agnieszka Kowalczyk, Ann Petersen, Goli Naderi-Oboodi, Lucj a Orzechowski, and Quentin Strebel. DNA Compatible Multistep Synthesis and Applications to DNA Encoded Libraries Bioconjugate Chemistry 2015 26 (8), 1623-1632; (2) Beatch, G. N., Liu, Y., and Plouvier, B. M. C. PCT Int. Appl. 2001096335, Dec. 20, 2001; (3) Inglis, S. R., Jones, R. K., Booker, G. W., and Pyke, S. M. (2006) Synthesis of N-benzylated-2-aminoquinolines as ligands for the Tec SH3 domain. Bioorg. Med. Chem. Lett. 16, 387-390.)

### Example 11: Synthesize an Encoded Portion Using Sonogashira Coupling Chemistry

A DNA library bearing an aryl-iodide, either as a reactive site on a reaction site adapter, as a building block on a charged reaction site adapter or as a partially translated molecule, is dissolved in water at 1 mM. To it is added 100 equivalents of an alkyne as a 200 mM stock solution in dimethylacetamide, 300 equivalents of pyrrolidine as a 200 mM stock solution in dimethylacetamide, 0.4 equivalents of palladium acetate as a 10 mM stock solution in dimethylacetamide, 2 equivalents of 3, 3′,3″ phosphinetriyltris (benzenesulfonic acid) trisodium salt as a 100 mM aqueous solution. The reaction is run for 2 hours at 65° C., then purified by ethanol precipitation or by ion exchange chromatography. (See (1) Liang, B., Dai, M., Chen, J., and Yang, Z. (2005) Cooper-free sonogashira coupling reaction with PdCl2 in water under aerobic conditions. J. Org. Chem. 70, 391-393; (2) Li, N., Lim, R. K. V., Edwardraja, S., and Lin, Q. (2011) Copper-free Sonogashira cross-coupling for functionalization of alkyne encoded proteins in aqueous medium and in bacterial cells. J. Am. Chem. Soc. 133, 15316-15319; (3) Marziale, A. N., Schlüter, J., and Eppinger, J. (2011) An efficient protocol for copper-free palladium-catalyzed Sonogashira crosscoupling in aqueous media at low temperatures. Tetrahedron Lett. 52, 6355-6358; (4) Kanan, M. W., Rozenman, M. M., Sakurai, K., Snyder, T. M., and Liu, D. R. (2004) Reaction discovery enabled by DNA-templated synthesis and in vitro selection. Nature 431, 545-549.)

### Example 12: Synthesize an Encoded Portion Using Carbamylation

A DNA library bearing an amine, either as a reactive site on a reaction site adapter, as a building block on a charged reaction site adapter or as a partially translated molecule, is dissolved in water at 1 mM. To it is added 1:4 v/v triethylamine, 50 equivalents of di-2-pyridylcarbonate as a 200 mM stock solution in dimethylacetamide. The reaction is run for 2 hours at room temp, then 40 equivalents of an amine as a 200 mM stock solution in dimethylacetamide is added at room temperature for 2 hours. The product is purified by ethanol precipitation, or ion exchange chromatography. (See (1) Artuso, E., Degani, I., and Fochi, R. (2007) Preparation of mono-, di-, and trisubstituted ureas by carbonylation of aliphatic amines with S,S-dimethyl dithiocarbonate. Synthesis 22, 3497-3506; (2) Franch, T., Lundorf, M. D., Jacobsen, S. N., Olsen, E. K., Andersen, A. L., Holtmann, A., Hansen, A. H., Sorensen, A. M., Goldbech, A., De Leon, D., et al. Enzymatic encoding methods for efficient synthesis of large libraries. WIPO WO 2007/062664 A2, 2007.)

### Example 13: Synthesize an Encoded Portion Using Thioureation

A DNA library bearing an amine, either as a reactive site on a reaction site adapter, as a building block on a charged reaction site adapter or as a partially translated molecule, is dissolved in water at 1 mM. To it is added 20 equivalents 2-pyridylthionocarbonate as a 200 mM stock solution in dimethylacetamide at room temperature for 30 minutes. Then 40 equivalents of an amine are added as a 200 mM stock solution in dimethylacetamide at room temperature and slowly warmed to 60 μC and reacted for 18 hours. The product is purified by ethanol precipitation, or ion exchange chromatography. (See Deprez-Poulain, R. F., Charton, J., Leroux, V., and Deprez, B. P. (2007) Convenient synthesis of 4H-1,2,4-triazole-3-thiols using di-2-pyridylthionocarbonate. Tetrahedron Lett. 48, 8157-8162.)

### Example 14: Synthesize an Encoded Portion Using Reductive Mono-Alkylation of an Amine

A DNA library bearing an amine, either as a reactive site on a reaction site adapter, as a building block on a charged reaction site adapter or as a partially translated molecule, is dissolved in water at 1 mM. To it is added 40 equivalents of aldehyde as a 200 mM stock in dimethylacetamide, and reacted at room temperature for 1 hour. Then 40 equivalents of sodium borohydride are added as a 200 mM stock solution in acetonitrile and reacted at room temperature for 1 hour. The product is purified by ethanol precipitation, or ion exchange chromatography. (See Abdel-Magid, A. F., Carson, K. G., Harris, B. D., Maryanoff, C. A., and Shah, R. D. (1996) Reductive amination of aldehydes and ketones with sodium triacetoxyborohydride. J. Org. Chem. 61, 3849-3862.)

### Example 15: Synthesize an Encoded Portion Using SNAr with Heteroaryl Compounds

A DNA library bearing an amine, either as a reactive site on a reaction site adapter, as a building block on a charged reaction site adapter or as a partially translated molecule, is dissolved in water at 1 mM. To it is added 60 equivalents of a heteroarylhalide as a 200 mM stock solution in dimethylacetamide and reacted at 60° C. for 12 hours. The product is purified by ethanol precipitation, or ion exchange chromatography. (See Franch, T., Lundorf, M. D., Jacobsen, S. N., Olsen, E. K., Andersen, A. L., Holtmann, A., Hansen, A. H., Sorensen, A. M., Goldbech, A., De Leon, D., et al. Enzymatic encoding methods for efficient synthesis of large libraries. WIPO WO 2007/062664 A2, 2007.)

### Example 16: Synthesize an Encoded Portion Using Horner-Wadsworth-Emmons Chemistry

A DNA library bearing an aldehyde, either as a reactive site on a reaction site adapter, as a building block on a charged reaction site adapter or as a partially translated molecule, is dissolved in borate buffer pH 9.4 at 1 mM. To it is added 50 equivalents of ethyl 2-(diethoxyphsophoryl)acetate as a 200 mM stock in dimetylacetamide, and 50 equivalents of cesium carbonate as a 200 mM aqueous solution and reacted at room temperature for 16 hours. The product is purified by ethanol precipitation, or ion exchange chromatography. (See Manocci, L., Leimbacher, M., Wichert, M., Scheuermann, J., and Neri, D. (2011) 20 years of DNA-encoded chemical libraries. Chem. Commun. 47, 12747-12753.)

### Example 17: Synthesize an Encoded Portion Using Sulfonylation

A DNA library bearing an amine, either as a reactive site on a reaction site adapter, as a building block on a charged reaction site adapter or as a partially translated molecule, is dissolved in borate buffer pH 9.4 at 1 mM. To it is added 40 equivalents of a sulfonyl chloride as a 200 mM stock solution in dimethylacetamide and reacted at room temp for 16 hours. The product is purified by ethanol precipitation, or ion exchange chromatography. (See Franch, T., Lundorf, M. D., Jacobsen, S. N., Olsen, E. K., Andersen, A. L., Holtmann, A., Hansen, A. H., Sorensen, A. M., Goldbech, A., De Leon, D., et al. Enzymatic encoding methods for efficient synthesis of large libraries. WIPO WO 2007/062664 A2, 2007.)

### Example 18: Synthesize an Encoded Portion Using Trichloro-Nitro-Pyrimidine

A DNA library bearing an amine, either as a reactive site on a reaction site adapter, as a building block on a charged reaction site adapter or as a partially translated molecule, is dissolved in borate buffer pH 9.4 at 1 mM. To it is added 20 equivalents of trichloro-nitro-pyrimidine (TCNP) as a 200 mM stock solution in dimethylacetamide at 5° C. The reaction is warmed to room temp over an hour and purified by ethanol precipitation. The DNA library is dissolved at 1 mM in borate buffer pH 9.4 and 40 equivalents of amine are added as a 200 mM stock solution in dimethylacetamide, 100 equivalents of neat triethylamine and reacted at room temp for 2 hours. The library is purified by ethanol precipitation. The DNA library is either immediately dissolved in borate buffer for immediate reaction, or it is pooled, re-sorted on an array and then dissolved in borate buffer, whereupon it is reacted with 50 equivalents of an amine as a 200 mM stock in dimtheylacetamide and 100 equivalents of triethylamine and reacted at room temperature for 24 hours. The product is purified by ethanol precipitation, or ion exchange chromatography. (See Roughley, S. D., and Jordan, A. M. (2011) The medicinal chemist's toolbox: an analysis of reactions used in the pursuit of drug candidates. J. Med. Chem. 54, 3451-3479.)

### Example 19: Synthesize an Encoded Portion Using Trichloropyrimidine

A DNA library bearing an amine, either as a reactive site on a reaction site adapter, as a building block on a charged reaction site adapter or as a partially translated molecule, is dissolved in borate buffer pH 9.4 at 1 mM. To it is added 50 equivalents of 2,4,6 trichloropyrimidine as a 200 mM stock in DMA and reacted at room temp for 3.5 hours. The DNA is precipitated in ethanol, and then re-dissolved in borate buffer pH 9.4 at 1 mM. To it is added 40 equivalents of amine as a 200 mM acetonitrile stock and reacted at 60-80° C. for 16 hrs. The product is purified by ethanol precipitation and then the DNA library is either immediately dissolved in borate buffer for immediate reaction, or it is pooled, re-sorted on an array and then dissolved in borate buffer, whereupon it is reacted with 60 equivalents of a boronic acid as a 200 mM stock in dimethylacetamide (DMA) and 200 equivalents of sodium hydroxide as a 500 mM aqueous solution, 2 equivalents of palladium acetate as a 10 mM DMA stock and 20 equivalents of tris(3-sulfophenyl)phosphine trisodium salt (TPPTS) as a 100 mM aqueous solution, and reacted at 75° C. for 3 hours. The DNA is precipitated in ethanol, then dissolved in water at 1 mM and reacted with 120 equivalents of sodium sulfide as a 400 mM stock in water at 65° C. for 1 hour. The product is purified by ethanol precipitation, or ion exchange chromatography.

### Example 20: Synthesize an Encoded Portion Using Boc-Deprotection

A DNA library bearing a Boc-protected amine, as a reactive site on a reaction site adapter, as a building block on a charged reaction site adapter, or as a partially translated molecule, is dissolved in borate buffer pH 9.4 at 0.5 mM, and heated to 90° C. for 16 hours. The product is purified by ethanol precipitation, size exclusion chromatography or ion exchange chromatography. (See Franch, T., Lundorf, M. D., Jacobsen, S. N., Olsen, E. K., Andersen, A. L., Holtmann, A., Hansen, A. H., Sorensen, A. M., Goldbech, A., De Leon, D., et al. Enzymatic encoding methods for efficient synthesis of large libraries. WIPO WO 2007/062664 A2, 2007.)

### Example 21: Synthesize an Encoded Portion Using Hydrolysis of a t-Butyl Ester

A DNA library bearing t-butyl ester, as a reactive site on a reaction site adapter, as a building block on a charged reaction site adapter, or as a partially translated molecule, is dissolved in borate buffer at 1 mM, and reacted at 80° C. for 2 hours. The product is purified by ethanol precipitation, size exclusion chromatography or ion exchange chromatography. (See Franch, T., Lundorf, M. D., Jacobsen, S. N., Olsen, E. K., Andersen, A. L., Holtmann, A., Hansen, A. H., Sorensen, A. M., Goldbech, A., De Leon, D., et al. Enzymatic encoding methods for efficient synthesis of large libraries. WIPO WO 2007/062664 A2, 2007.)

### Example 22: Synthesize an Encoded Portion Using Alloc-Deprotection

A DNA library bearing an Alloc-protected amine, as a reactive site on a reaction site adapter, as a building block on a charged reaction site adapter or as a partially translated molecule, is dissolved in borate buffer pH 9.4 at 1 mM. To it is added 10 equiv. of palladium tetrakis triphenylphosphine as a 10 mM DMA stock, and 10 equiv. of sodium borohydride as a 200 mM acetonitrile stock and reacted at room temperature for 2 hours. The product is purified by ethanol precipitation, or ion exchange chromatography. (See Beugelmans, R., Neuville, M. B.-C., Chastanet, J., and Zhu, J. (1995) PalladiμM catalyzed reductive deprotection of Alloc: Transprotection and peptide bond formation. Tetrahedron Lett. 36, 3129.)

### Example 23: Synthesize an Encoded Portion Using Hydrolysis of a Methyl/Ethyl Ester

A DNA library bearing methyl or ethyl ester, as a reactive site on a reaction site adapter, as a building block on a charged reaction site adapter, or as a partially translated molecule, is dissolved in borate buffer at 1 mM, and reacted with 100 equiv of NaOH at 60° C. for 2 hours. The product is purified by ethanol precipitation, size exclusion chromatography or ion exchange chromatography. (See Franch, T., Lundorf, M. D., Jacobsen, S. N., Olsen, E. K., Andersen, A. L., Holtmann, A., Hansen, A. H., Sorensen, A. M., Goldbech, A., De Leon, D., et al. Enzymatic encoding methods for efficient synthesis of large libraries. WIPO WO 2007/062664 A2, 2007.)

### Example 24: Synthesize an Encoded Portion Using Reduction of a Nitro Group

A DNA library bearing a nitro group, as a reactive site on a reaction site adapter, as a building block on a charged reaction site adapter, or as a partially translated molecule, is dissolved in water at 1 mM. To it is added 10% volume equiv. of Raney nickel slurry, 10% volume equiv. of hydrazine as a 400 mM aqueous solution and reacted at room temp for 2-24 hrs with shaking. The product is purified by ethanol precipitation, or ion exchange chromatography. (See Balcom, D., and Furst, A. (1953) Reductions with hydrazine hydrate catalyzed by Raney nickel. J. Am. Chem. Soc. 76, 4334-4334.)

### Example 25: Synthesize an Encoded Portion Using “Click” Chemistry

A DNA library bearing an alkyne or an azide group, as a reactive site on a reaction site adapter, as a building block on a charged reaction site adapter or as a partially translated molecule, is dissolved in 100 mM phosphate buffer at 1 mM. To it is added copper sulfate to 625 μM, THPTA (ligand) to 3.1 mM, amino-guanidine to 12.5 mM, ascorbate to 12.5 mM, and an azide to 1 mM (if the DNA bears an alkyne) or an alkyne to 1 mM (if the DNA bears an azide). The reaction is run at room temperature for 4 hours. The product is purified by ethanol precipitation, size exclusion chromatography or ion exchange chromatography. (See Hong, V., Presolski, Stanislav I., Ma, C. and Finn, M. G. (2009), Analysis and Optimization of Copper-Catalyzed Azide-Alkyne Cycloaddition for Bioconjugation. Angewandte Chemie International Edition, 48: 9879-9883.)

### Example 26: Synthesize an Encoded Portion Incorporating a Benzimidazole

A DNA library bearing an aryl vicinal diamine, as a reactive site on a reaction site adapter, as a building block on a charged reaction site adapter or as a partially translated molecule, is dissolved in borate buffer pH 9.4 at 1 mM. To it is added 60 equiv. of an aldehyde as a 200 mM DMA stock and reacted at 60° C. for 18 hours. The product is purified by ethanol precipitation, or ion exchange chromatography. (See (1) Mandal, P., Berger, S. B., Pillay, S., Moriwaki, K., Huang, C., Guo, H., Lich, J. D., Finger, J., Kasparcova, V., Votta, B., et al. (2014) RIP3 induces apoptosis independent of pronecrotic kinase activity. Mol. Cell 56, 481-495; (2) Gouliaev, A. H., Franch, T. P.-O., Godskesen, M. A., and Jensen, K. B. (2012) Bi-functional Complexes and methods for making and using such complexes. Patent Application WO 2011/127933 A1; (3) Mukhopadhyay, C., and Tapaswi, P. K. (2008) Dowex 50W: A highly efficient and recyclable green catalyst for the construction of the 2-substituted benzimidazole moiety in aqueous medium. Catal. Commun. 9, 2392-2394.)

### Example 27: Synthesize an Encoded Portion Incorporating an Imidazolidinone

A DNA library bearing an alpha-amino-amide, either as a reactive site on a reaction site adapter, as a building block on a charged reaction site adapter or as a partially translated molecule, is dissolved in 1:3 methanol:borate buffer pH 9.4 at 1 mM. To it is added 60 equiv. of an aldehyde as a 200 mM DMA stock and reacted at 60° C. for 18 hours. The product is purified by ethanol precipitation, or ion exchange chromatography. (See (1) Barrow, J. C., Rittle, K. E., Ngo, P. L., Selnick, H. G., Graham, S. L., Pitzenberger, S. M., McGaughey, G. B., Colussi, D., Lai, M.-T., Huang, Q., et al. (2007) Design and synthesis of 2,3,5-substituted imidazolidin-4-one inhibitors of BACE-1. Chem. Med. Chem. 2, 995-999; (2) Wang, X.-J., Frutos, R. P., Zhang, L., Sun, X., Xu, Y., Wirth, T., Nicola, T., Nummy, L. J., Krishnamurthy, D., Busacca, C. A., Yee, N., and Senanayake, C. H. (2011) Asymmetric synthesis of LFA-1 inhibitor BIRT2584 on metric ton scale. Org. Process Res. Dev. 15, 1185-1191; (3) Blass, B. E., Janusz, J. M., Wu, S., Ridgeway, J. M. II, Coburn, K., Lee, W., Fluxe, A. J., White, R. E., Jackson, C. M., and Fairweather, N. 4-Imidazolidinones as KV1.5 Potassium channel inhibitors. WIPO WO2009/079624 A1, 2009.)

### Example 28: Synthesize an Encoded Portion Incorporating a Quinazolinone

A DNA library bearing an 2-anilino-1-benzamide, either as a reactive site on a reaction site adapter, as a building block on a charged reaction site adapter or as a partially translated molecule, is dissolved in borate buffer pH 9.4 at 1 mM. To it is added 200 equiv. NaOH as a 1M solution in water and an aldehyde as a 200 mM stock solution in DMA and reacted at 90° C. for 14 hours. The product is purified by ethanol precipitation, or ion exchange chromatography. (See Witt, A., and Bergmann, J. (2000) Synthesis and reactions of some 2-vinyl-3H-quinazolin-4-ones. Tetrahedron 56, 7245-7253.)

### Example 29: Synthesize an Encoded Portion Incorporating an Isoindolinone

A DNA library bearing an amine, either as a reactive site on a reaction site adapter, as a building block on a charged reaction site adapter or as a partially translated molecule, is dissolved in borate buffer pH 9.4 at 1 mM. To it is added a 4-bromo, 2-ene methyl ester as a 200 mM stock solution in DMA and reacted for 2 hours at 60° C. The product is purified by ethanol precipitation, or ion exchange chromatography. (See Chauleta, C., Croixa, C., Alagillea, D., Normand, S., Delwailb, A., Favotb, L., Lecronb, J.-C., and Viaud-Massuarda, M. C. (2011) Design, synthesis and biological evaluation of new thalidomide analogues as TNF-α and IL-6 production inhibitors. Bioorg. Med. Chem. Lett. 21, 1019-1022.)

### Example 30: Synthesize an Encoded Portion Incorporating a Thiazole

A DNA library bearing a thiourea, either as a reactive site on a reaction site adapter, as a building block on a charged reaction site adapter or as a partially translated molecule, is dissolved in borate buffer pH 9.4 at 1 mM. To this is added 50 equiv. of a bromoketone as a 200 mM stock in DMA and reacted at room temp for 24 hours. The product is purified by ethanol precipitation, or ion exchange chromatography. (See Potewar, T. M., Ingale, S. A., and Srinivasan, K. V. (2008) Catalyst-free efficient synthesis of 2-aminothiazoles in water at ambient temperature. Tetrahedron 64, 5019-5022.)

### Example 31a: Synthesize an Encoded Portion Using Various Other Chemistries

Thirty-one types of compatible chemical reactions are listed with references in Handbook for DNA-Encoded Chemistry (Goodnow R. A., Jr., Ed.) pp 319-347, 2014 Wiley, New York. These include SNAr reactions of trichlorotriazines, diol oxidations to glyoxal compounds, Msec deprotection, Ns deprotection, Nvoc deprotection, pentenoyl deprotection, indole-styrene coupling, Diels-Alder reaction, Wittig reaction, Michael addition, Heck reaction, Henry reaction, nitrone 1,3-dipolar cycloaddition with activated alkenes, formation of oxazolidines, trifluoroacetamide deprotection, alkene-alkyne oxidative coupling, ring-closing metatheses and aldol reactions. Other reactions are published in this reference that have the potential of working in the presence of DNA and are appropriate for use.

### Example 31b. Chemistries for Charging Reaction Site Adapters

It is understood that any of the chemistries described in Examples 9-31 are appropriate for use in charging a reaction site adapter. Reaction site adapters are charged with a building block in aqueous solution, in aqueous/organic mixtures, or when immobilized on a solid support. The chemistry used to charge a reaction site adapter with a building block is not limited to reactions performed while the reaction site adapter is immobilized on a solid support like DEAE or Super Q650M; nor is it limited to reactions carried out in solution phase.

### Example 32: Use Different Restriction Enzymes in Library Preparation

It will be understood that the restriction enzymes named in other Examples are representative, and that other restriction enzymes may serve the same purpose with equanimity or advantage.

### Example 33. Performing Selections for Binding a Target Molecule Using an Alternative Method

Selections to identify library members capable of binding a target molecule are performed as per Example 5 with the exception that target molecules are immobilized on the surface of plastic plates like IMMULON® plates, MAXISORP® plates or other plates commonly used for immobilizing biological macromolecules for ELISA, or the target molecules are biotinylated and immobilized on streptavidin-coated surfaces or neutravidin-coated surfaces, or avidin-coated surfaces, including magnetic beads, beads made of synthetic polymers, beads made of polysaccharides or modified polysaccharides, plate wells, tubes, and resins. It will be understood that selections to identify library members possessing a desired trait will be performed in buffers that are compatible with DNA, compatible with keeping any target molecules in a native conformation, compatible with any enzymes used in the selection or amplification process, and compatible with identification of trait-positive library members. Such buffers include, but are not limited to, buffers made with phosphate, citrate, and TRIS. Such buffers may also include, but not be limited to, salts of potassium, sodium, ammonium, calcium, magnesium and other cations, and chloride, iodide, acetate, phosphate, citrate, and other anions. Such buffers may include, but not be limited to, surfactants like TWEEN®, TRITON™, and Chaps (3-[(3-Cholamidopropyl)dimethylammonio]-1-propanesulfonate).

### Example 34. Selection for Binders with Long Off-Rates

Selections are performed to identify individuals in the library population possessing the ability to bind a target molecule as described in Example 5. Individuals that bind the target molecule with long off-rates are selected for as follows. Target molecules are immobilized by being biotinylated and incubated with a streptavidin-coated surface, or in some cases, immobilized without biotinylation on plastic surface like a MAXISORP® plate or some other plate suitable for binding proteins for ELISA-like assays, or by a method described in Example 35, or by another method. The library population is incubated with the immobilized target for 0.1 to 8 hours in an appropriate buffer. The duration of the incubation will depend on the estimated number of copies of each individual library member in the sample and on the number of target molecules immobilized. With higher copy numbers of individuals and higher loads of target molecule, the duration may diminish. With smaller copy numbers and/or smaller loads of a target molecule, the duration may extend. An objective is to ensure each individual in the population has the opportunity to fully interact with the target. After this incubation of the library with an immobilized target, binders in the library are presumed to be bound to the target. At this point, an excess of non-immobilized target is added to the system and the incubation is continued for about 1 to about 24 hours. Any individuals bound to an immobilized target that possesses a short off-rate, may release from the immobilized target and upon re-binding will partition into being bound by free target and immobile target. Individuals binding with long off-rates will remain bound to the immobilized target. Washing the immobilization surface preferentially removes non-binders and binders with fast off-rates, thus selecting for individuals with long off-rates. Amplification of the DNA encoding the long off-rate binders is done as above in Example 3 and 5.

### Example 35. Selections with Mobile Targets

Selections are performed in which target molecules are biotinylated, and then incubated with a library for an appropriate duration. The mixture is then immobilized for example on a streptavidin surface, whereupon the target becomes immobilized, and any library members bound to the target become immobilized as well. Washing the surface removes non-binders. Amplification of the DNA encoding the binders is done as above.

### Example 36. Selections for Target Specificity

Selections are performed to identify individuals in the library population that bind to a desired target molecule to the exclusion of other anti-target molecules. The anti-target molecule (or molecules if there are more than one) are biotinylated and immobilized on a streptavidin-coated surface, or in some cases, immobilized on a plastic surface like a MAXISORP® plate or some other plate suitable for binding proteins for ELISA-like assays. In a separate container, target molecules are immobilized by being biotinylated and incubated with a streptavidin-coated surface, or in some cases, immobilized on plastic surface like a MAXISORP® plate or some other plate suitable binding proteins for ELISA-like assays. The library is first incubated with the anti-target. This depletes the population of individuals that bind the anti-target molecule(s). After this incubation with anti-target, the library is transferred to a container with desired target and incubated for an appropriate duration. Washing removes non-binders. Amplification of the DNA encoding the long off-rate binders is done as per Example 1. Target binders identified will have an improved probability of selectively binding the target over the anti-target(s). In some cases, the selection for affinity for a target is performed by immobilizing the target, adding free, mobile anti-target in excess, and then adding library and incubating for an appropriate duration. Under this regime, individuals with affinity for the anti-target are preferentially bound by the anti-target because it is present in excess, and can thus be removed during washing of the surface. Amplification of the DNA encoding the binders is done as per Example 3 and 5.

### Example 37. Selections Based on Differential Mobility

Selections are performed based on the ability of an individual in the library population to interact with a target molecule or polymacromolecular structure based on a difference in mobility of the library member when in a complex formed when a target molecule or polymacromolecular structure is interacting with the library member. Allowing target molecules or structures and library members to interact, and then passing the mixture through a size exclusion medium causes library members that are not interacting with a target molecule or structure to become physically separated from library members that are interacting, because the complex of the interacting library member and target molecule or structure will be larger than non-interacting library members, and therefore move through the medium with a different mobility. It will be appreciated that the difference in mobility can be a function of diffusion in the absence of a size exclusion medium, that the mobility can be induced by various methods including but not limited to gravity flow, electrophoresis, and diffusion.

### Example 38. General Strategies for Other Selections

It will be appreciated by one skilled in the art that selections are performed for virtually any property provided an assay is designed that either (a) physically separates individuals in the library population that possess the desired property from individuals that do not possess it, or (b) allows DNA encoding individuals in the library population that possess the desired property to be preferentially amplified over DNA encoding library members that do not possess the property. Many methods of immobilization of target molecules are suitable, including tagging target molecules with His-tags and immobilizing on nickel surfaces, tagging target molecules with flag tags and immobilizing with anti-flag antibodies, or tagging target molecules with a linker and covalently immobilizing it to a surface. It will be appreciated that the order of the events that allow library members to bind targets and that allows targets to be immobilized is done in various orders as is dictated or enabled by the method of immobilization used. It will be appreciated that selections are performed wherein immobilization or physical separation of trait-positive individuals from trait-negative individuals is not required. For example, trait-positive individuals recruit factors enabling amplification of their DNA, where trait-negative members do not. Trait-positive individuals become tagged with a PCR primer, whereas trait-negative individuals do not. Any process differentially amplifying trait-positive individuals is suitable for use.

### Example 39. The Absence of a Building Block is an Encode-Able Diversity Element

In the course of library synthesis, diversity is generated when a multiplicity of building blocks are installed independently on various library subpools possessing different sequences. The absence of a building block is an optional diversity element. The absence of a building block is encoded exactly as per Examples 1-4, except that at a desired chemical step, one or more sequence-specific sub-pools of the library are not treated with any chemistry to install a building block. In such case the sequence of those sub-pools thereby encode the absence of a building block.

### Example 40. Hybridization Arrays Comprised of Other Materials

Hybridization Arrays can accomplish 2 critical tasks: (a) they can sort a heterogeneous mixture of at least partially single-stranded DNAs through sequence specific hybridization, and (b) the arrays can enable or allow the sorted sub-pools to be removed from the array independently. The features of the array wherein anti-coding oligonucleotides are immobilized may be arranged in any three dimensional orientation that meets the above criteria, but a 2 dimensional rectangular grid array is currently most attractive because an abundance of commercially available labware is already mass produced in that format (e.g. 96-well plates, 384-well plates).

The solid supports in the features of the array upon which anti-coding oligos are immobilized can accomplish 4 tasks: (a) it can permanently affix the anti-coding oligo, (b) it can enable or allow capture of a library DNA through sequence specific hybridization to the immobilized oligo, (c) it can have low background or non-specific binding of library DNA, and (d) it can be chemically stable to the processing conditions, including a step performed at high pH. CM SEPHAROSE® has been functionalized with azido-PEG-amine (with 9 PEG units) by peptide bond formation between the amine of azido-PEG-amine and carboxyl groups on the surface of the CM SEPHAROSE® resin. Anti-coding oligos bearing an alkynyl-modifier are ‘clicked’ to the azide in a copper-mediated 1,3-dipolar cycloaddition (Huisgen).

Other suitable solid supports include hydrophilic beads, or polystyrene beads with hydrophilic surface coatings, polymethylmethacrylate beads with hydrophilic surface coatings, and other beads with hydrophilic surfaces which also bear a reactive functional group like a carboxylate, amine, or epoxide, to which an appropriately functionalized anti-coding oligo is immobilized. Other suitable supports include monoliths and hydrogels. See, for example, J Chromatogr A. 2002 Jun. 14; 959(1-2):121-9, J Chromatogr A. 2011 Apr. 29; 1218 (17): 2362-7, J Chromatogr A. 2011 Dec. 9; 1218(49):8897-902, Trends in Microbiology, Volume 16, Issue 11, 543-551, J. Polym. Sci. A Polym. Chem., 35: 1013-1021, J. Mol. Recognit. 2006; 19: 305-312, J. Sep. Sci. 2004, 27, 828-836. Generally, solid supports with greater surface area capture a greater amount of library DNA, and beads with smaller diameter engender far higher back pressures and resistance to flow. These constraints are in part improved by the use of porous supports or hydrogels which have very high surface areas, but lower backpressures. Generally, beads with positive charges engender greater degrees of non-specific binding of DNA.

The chassis of the hybridization array can accomplish 3 tasks: (a) it must maintain the physical separation between features, (b) enable or allow a library to flow over or through the features, and (c) enable or allow removal of the sorted library DNA from different features independently. The chassis is comprised of any material that is sufficiently rigid, chemically stable under processing conditions, and compatible with any methods that are required for immobilizing supports within features. Typical materials for the chassis include plastics like DELRIN®, TECAFROM®, or polyether ether ketone (PEEK), ceramics, and metals, like aluminum or stainless steel.

### Example 41. Prepare Molecules of Formula (IV), and Formula (II) by Sorting a Library of Oligonucleotide G into a First Set of Sub-Pools, then Sorting Each Sub-Pool into a Second Set of Sub-Pools, then Sorting Each Sub-Pool into an Nth Set of Sub-Pools and Performing Chemistry Specific to Each Sub-Pool

Three or more independent coding regions can be used to encode a building block. Under such a regime, a library is prepared as described above, and hybridization arrays or columns are prepared as above. The library is then sorted on a first array by a first coding region to produce a set of sub-pools. Each sub-pool is then sorted on a second array into a second set of sub-pools, each of which can be further sorted into a third set of sub-pools, whereupon chemistry is done to install building blocks or diversity elements in a sub-pool specific manner. Under this regime, 3 coding regions are used to encode a single diversity element or building block. For example, a library may be prepared as above with 16 coding sequences at each of 6 coding regions, and hybridization arrays prepared for each coding region. The library could be sorted into 16 sub-pools based on the coding sequences of the coding region nearest the 5′ end. Doing so would produce 16 sub-pools. Each of these sub-pools could be themselves sorted into 16 sub-pools based on the coding sequences of the coding region nearest the 3′ end (or any other predetermined coding region). Doing so would produce 256 sub-pools. Each of these sub-pools could be further sorted into 16 sub-pools based on the coding sequences of the coding region second from the 3′ end (or any other predetermined coding region). Doing so would produce 4096 sub-pools. Each of these sub-pools will be sorted based on three coding sequences, and different chemistry can be done at each of them. Subsequently, the library may be pooled and sorted into a second set of 4096 sub-pools by sorting sequentially on the 3 remaining coding regions in a predetermined order. Whereupon, different independent chemistries can be done as per Examples 9-31 in a sub-pool specific manner.

### Example 42. Preparation of Libraries with Different Numbers of Coding Sequences and Coding Regions

It will be appreciated that (a) the number of coding sequences at any given coding region can vary, (b) the number of coding regions in a designed library can vary, (c) the number of coding sequences at different coding regions can be the same or different, and (d) that a library can be prepared in which a single coding region encodes some building blocks, and multiple coding regions encode other building blocks.

For example, a library can be prepared with 5 coding regions, in which there are 32 coding sequences at each of 2 coding regions, 96 coding sequences at a 3^{rd} region, 2 coding sequences at a 4th region, and 1536 coding sequences at a 5^{th} region. It will also be appreciated by one skilled in the art that the order in which coding regions are used for sorting can vary from implementation of the library to implementation, but that the order would be decided in advance, and used exclusively during each independent implementation, in order to retain the sequence-to-encoded molecule correspondence required for proper decoding and analysis of results.

### Example 43. Preparation of Libraries Using Both Mononomial and Multinomial Encoding

A library is prepared with 5 coding regions, in which there are 32 coding sequences at each of 2 coding regions, 96 coding sequences at a 3^{rd} region, 2 coding sequences at a 4^{th} region, and 768 coding sequences at a 5^{th} region.

The library is prepared as in Example 2c or purchased as in Example 2d. The library is prepared for translation as in Example 3. The library is translated as in Example 4, except that the library is sorted into 1024 sub-pools by sorting on an array with capture oligos complementary to the first coding region possessing 32 coding sequences, followed by sorting on a second array possessing 32 coding sequences complementary to the second coding region possessing 32 sequences, whereupon sub-pool specific chemistry is done as per Example 4e, or Example 9 through Example 31. In this manner two coding regions are required to encode a single building block.

The library is then pooled and sorted into 768 sub-pools by sorting on an array, or arrays, possessing 768 capture oligos complementary to the coding region possessing 768 coding sequences. Whereupon sub-pool specific chemistry is done as per Example 4e, or Example 9 through Example 31. In this manner one coding region is required to encode a single building block.

The library is then pooled and sorted into 192 sub-pools by sorting first on an array bearing 2 oligos complementary to the coding region possessing 2 coding sequences, and then sorted on a second array possessing 96 coding sequences. Whereupon sub-pool specific chemistry is done as per Example 4e, or Example 9 through Example 31. In this manner two coding regions are required to encode a single building block.

It will be appreciated by one skilled in the art that the number of sequences at each coding region can vary, and that the number of coding regions can also vary.

### Example 44. Alternative Method of Preparation of Libraries Using Both Mononomial and Multinomial Encoding

A library is prepared with 5 coding regions, in which there are 1536 coding sequences at each of 2 terminal coding regions, 2 coding sequences at a 3^{rd} region, 8 coding sequences at a 4^{th} region, and 96 coding sequences at a 5^{th} region. The library is prepared as in Example 2c or purchased as in Example 2d. The library is prepared for translation as in Example 3, with the following exceptions.

### Example 44a. Removal of Terminal Non-Coding Regions

The ssDNA product of the reverse-transcription reacted with complementary oligos making the non-coding regions double stranded is suspended in NEB CUTSMART® Buffer at a concentration of 100 μg/ml. Restriction enzymes, SACI-HF®, and ECORI-HF® from NEB are added to a concentration of 1 U/μg of DNA. The digestion is incubated for 1 hour at 37° C., and then the enzymes are heat inactivated at 65° C. for 20 minutes.

### Example 44b. Provision of Reaction Site Adapters

Two sets of 1536 reaction site adapters are provided, each comprising an anti-coding sequence and, in some cases, comprising a hairpin loop, and a stem with an overhang forming the said anti-coding sequence. One set has a 3′ anti-coding sequence that specifically hybridizes to the 3′ terminal coding region of the template strand as it appears after removal of the 3′ terminal non-coding region; the other set has a 5′ anti-coding sequence that specifically hybridizes to the 5′ terminal coding region of the template strand as it appears after removal of the 5′ terminal non-coding region. The set bearing 3′ anti-coding sequences are provided with 5′ phosphoryl groups. In this example, the stem region of each set possesses the same sequence of the corresponding terminal non-coding region removed by restriction digestion previously. The loop regions of each set bear a base modified with a linkered reactive site, N4-TriGl-Amino 2′deoxycytidine (from IBA, Goettingen, Germany). Adapters as described here can be purchased from DNA oligo synthesis companies like Sigma Aldrich, Integrated DNA Technologies of Coralville, Iowa, or Eurofins MWG of Louisville, Ky.

### Example 44c. Charging of Reaction Site Adapters

The two sets of 1536 reaction site adapters are provided in separate wells, and dissolved in TE buffer (Promega, Mass.). 15 μl of TOYOPEARL® SuperQ-650M (Sigma-Aldrich, St. Louis, Mo.) ion exchange resin is placed in each well of a filter plate and washed with 100 μl of 10 mM HOAc. Aliquots of each reaction site adapter proportionate to the amount of template strand are transferred into separate wells of the filter plate wherein they are immobilized on the resin. The adapters immobilized on the resin are washed with dH₂O, then with piperidine, then with dimethylformamide (“DMF”). 2×1536 reaction solutions are made separately, each containing: 50 μl of DMF, an Fmoc-protected amino acid at 75 mM, 4-(4,6-Dimethoxy-1,3,5-triazin-2-yl)-4-methyl morpholinium tetrafluoroborate at 75 mM, N-methyl morpholine at 90 mM. These mixtures are allowed to activate the acid for ten minutes at room temp then added to the resin and reacted for 30 minutes. The resin is then washed 4×100 μl with DMF, and the coupling step repeated with a freshly prepared reaction mixture, washed again with DMF, and the Fmoc protecting group is removed by adding 50 μl of 20% piperidine in DMF to each well and incubating for 2 hours at room temperature. The resin is washed again 4×100 μl with DMF, then 3×100 μl with dH₂O. The charged reaction site adapters are eluted off the resin with 1.5 M NaCl, 50 mM KOH, 0.01% TRITON™ X-100. The solution is neutralized by addition of Tris to 15 mM and HOAc to pH 7.4. The charged reaction site adapters are then pooled and desalted by passing over a ZEBA™ 7K MWCO (Thermo Fisher Scientific, MA) desalting cartridge. Alternatively, the reaction may be conducted as above except that it is conducted in a mixture of DMF and water in solution phase in the absence of an ion exchange resin, and is purified by ethanol precipitation.

### Example 44d. Ligation of Charged Reaction Site Adapters to the Library

The restriction digested template library is buffer exchanged using ZEBA™30K MWCO (Thermo Fisher Scientific, MA) centrifugal concentrators to 50 mM Tris-HCl, 10 mM MgCl₂, 25 mM NaCl, pH 7.5@25° C. 1.1 equivalents of charged reaction site adapters specific for the 3′ end of the template strand are added; 1.1 equivalents of charged reaction site adapters specific for the 5′ end of the template strand are added, and the mixture is diluted with the same buffer to a template strand concentration of 1 μM. The reaction is warmed to 65° C. for 10 min and allowed to cool to 45° C. over 1 hr, and held at 45° C. for 4 hours. After cooling to room temperature, DTT is added to 10 mM, ATP is added to 1 mM, and T4 DNA Ligase is added to 50 U/mL. The ligation reaction is run at room temp for 12 hours, then the enzyme is heat inactivated at 65° C. for 10 min, and the reaction cooled slowly to room temperature. The reaction is buffer exchanged and concentrated with a 30K molecular weight cut-off (MWCO) centrifugal concentrator into 150 mM NaCl, 20 mM citrate, 15 mM Tris, 0.02% sodium dodecyl sulfate (“SDS”), 0.05% Tween20 (from Sigma-Aldrich), pH 7.5.

### Example 44e. Completion of Translation

The library is sorted as described above into 2 sub-pools on arrays bearing capture oligos complementary to sequences of the coding region possessing 2 coding sequences; then sorted into 16 sub-pools by sorting on arrays bearing capture oligos complementary to sequences of the coding region possessing 8 coding sequences; then sorted into 768 sub-pools by sorting a third time on arrays bearing complementary oligos to the sequences of the coding region possessing 96 coding sequences. Whereupon sub-pool specific chemistry is done as described in Example 4e, or in Examples 9-31.

Such a library would possess ˜2.4 million members. There would be 1536×768 combinations of building blocks at the 5′ end and a different combination of 1536×768 building blocks at the 3′ end. Although the building blocks encoded multinomially by the interior coding regions would be the same in the encoded molecules at either end of oligonucleotide G, the building blocks encoded mononomially by the two terminal coding regions would only be the same in 1 out of every 1536 library members.

### Example 45. Prepare and Translate a Library with a Single Reaction Site Adapter

A library with a single reaction site adapter is prepared exactly as per Example 44 with the exception that the steps of (a) removal of one of the terminal non-coding regions, and (b) ligation of the corresponding reaction site adapter, are omitted. For example, to prepare a library with a single reaction site adapter at the 3′ end of the coding strand of G, the library may be prepared exactly above, except that the only restriction endonuclease added should be EcoRI. Doing so will remove the 3′ terminal non-coding region, so that the 3′ charged reaction site adapters can appropriately hybridize and ligate to the template strand. Using only the restriction endonuclease specific for a recognition site in the 3′ terminal non-coding region and omitting the restriction endonuclease specific for a recognition site in the 5′ terminal non-coding region will leave the 5′ terminal non-coding region in place, disallowing ligation of 5′ reactive site adapters to that terminus. Addition of the 3′ charged reaction site adapters is done as above. Addition of the 5′ charged reactive site adapters is omitted. It will be appreciated by one skilled in the art that other restriction sites could be designed into the 3′ terminal coding region, and that different restriction enzymes could be used for this purpose.

### Example 46a. Alternative Method to Prepare and Translate a Library with a Single Reaction Site Adapter at the 5′ End

A library is prepared as in Example 2c or purchased as in Example 2d with the exception that a BsaI restriction site is used in the 5′ terminal non-coding region of the coding strand at positions 14-19 from the 5′ end of the 20-base non-coding sequence. After PCR of the library as in Example 3a, the terminal non-coding region at the 5′ end of the coding strand is removed by digestion with BsaI-HF from NEB as described in Example 1b. The library is transcribed as described above.

### Example 46b. Provision of Alternative Reaction Site Adapters

Alternative reaction site adapters are provided, each comprising a 5′ non-coding region, and a coding sequence. The non-coding region in some cases comprises a hairpin loop and a stem region. The coding sequence specifically hybridizes to the 3′ terminal coding region of the RNA template strand. In this example, the non-coding region of each adapter bears a base modified with a linkered reactive site, N4-TriGl-Amino 2′deoxycytidine (from IBA, Goettingen, Germany) and an encoded building block. Adapters as described here can be purchased from DNA oligo synthesis companies like Sigma Aldrich, Integrated DNA Technologies of Coralville, Iowa, or Eurofins MWG of Louisville, Ky.

### Example 46c. Charging Alternative Reaction Site Adapters is as Described Above in Example 44c

### Example 46d. Installation of 5′ Reaction Site Adapters by Reverse Transcription

Reverse Transcription is conducted as described as in Example 3c, with the following exceptions. The charged reaction site adapters from Example 46 are used as the primers for the reverse transcription reaction.

### Example 47. Prepare and Translate a Library with Reaction Site Adapters Ligated at Different Points During Synthesis

A library with 2 reaction site adapters can be prepared in which one reaction site adapter is installed on the template oligonucleotide G, then one or more positional building blocks are installed on it, before a second reaction site adapter is then installed on G. Several regimes may be used to achieve this: a charged reaction site adapter can be installed at the 5′ end during reverse transcription as described in Example 46, then the library can be sorted into sub-pools by mononomial or multinomial encoding and chemical synthesis, then a charged (or uncharged) reaction site adapter can be installed at the 3′ end as described in Example 45.

### Example 48. Prepare and Translate a Library with 2 or More Reaction Sites Per Reaction Site Adapter

A library with multiple reaction sites on a single adapter can be prepared exactly as above with the exception that reaction site adapters are provided that bear 2 (or more) bases modified with reactive sites like those described in Example 44b. Several placements of the reactive site modified bases are possible, including placement of bases bearing reactive sites nearer or farther from each other in the reaction site adapter. Multiple reactive sites can be placed on an adapter when only one adapter is being used, or when two adapters are being used. Such reaction site adapters are synthesized or purchased from a DNA oligo synthesis company like IDT, of Coralville, Iowa, or Eurofins MWG of Louisville, Ky.

### Example 49. Prepare and Translate a Library with Alternate Hairpins in the Reaction Site Adapter

Numerous versions of hairpins can be made and used in various contexts with the same protocols described in Example 44-46. In cases where a smaller hairpin is advantageous, the stem can comprise as few as 5 base pairs. Also, hairpins comprised of a 6-PEG linker between the complementary stem sequences can replace the larger DNA loop. See, Durand, M., et al. “Circular dichroism studies of an oligodeoxyribonucleotide containing a hairpin loop made of a hexaethylene glycol chain: conformation and stability.” Nucleic acids research 18.21 (1990): 6353-6359). For cases where polydisplay is advantageous, the distance between multiple encoded portions on a given hairpin, and the placement of those encoded portions, can be important, and can be designed in by rational placement of multiple bases bearing linkers at different locations in the adapter. For example, the distance between encoded portions is made larger or smaller by placing one linker in or near the loop region, keeping the number of nucleotides in the stem constant, but varying the location of the second linker along the length of the stem. Where the placement of the encoded portion on the hairpin is important, e.g., in cases where an encoded portion placed along a stem has different access to a target molecule than an encoded portion placed in a loop, hairpins with multiple loops and stems are used. In one embodiment, a hairpin may have 2 or 3 loops and 2 stems. This hairpin may comprise an anti-coding region connected to a first strand of a first stem region which is connected to a first loop region which is connected to a first strand of a second stem region which is connected to a second loop region which is connected to a second strand of the second stem region which is connected, in some cases, to a third loop region then to a second strand of the first stem region, or directly to a second strand of the first stem region. One or more linkers are placed in one or more of the loops, and in one or more of the stem regions as is needed for the particular project.

It will be appreciated by one skilled in the art that a great number of hairpin tertiary structures are possible which incorporate many secondary structures including, but not limited to, internal loops, bulges, and cruciform structures as described in Svoboda, P. et al. Cellular and Molecular Life Sciences CMLS, April 2006, Volume 63, Issue 7, pp 901-908, and in Bikard, et al., Microbiology And Molecular Biology Reviews, Dec. 2010, p. 570-588, and in Kari, et al., DNA Computing Volume 3892 of the series Lecture Notes in Computer Science pp 158-170, and in Domaratzki, Theory Comput Syst (2009) 44: 432-454, Brazda, et al, BMC Molecular Biology 2011 12:33. It will be appreciated by one skilled in the art that hairpin oligo sequences incorporating such secondary and tertiary structures are synthesized by many DNA synthesis companies like Sigma-Aldrich, Integrated DNA Technologies (of Coralville, Iowa), Eurofins MWG (of Louisville, Ky.). It will be appreciated that a modified base bearing a reactive site for installing a linker, or bearing a linker and a reactive site can be placed at any desirable locations in the hairpin during the course of synthesis. It will be appreciated by one skilled in the art that hairpins possessing more secondary structures and/or more information will tend to be comprised of longer nucleotide sequences.

### Example 50. Prepare and Translate a Library with Hairpins Possessing Other Functionalities in the Reaction Site Adapter

Numerous versions of the hairpins are made and used in various contexts with the same protocols described in Examples 44-49. The sequence of the stem region of the reaction site adapter can contain one or more restriction sites to allow cleavage in or near the stem region. This may enable release of very tight binders to immobilized targets, and facilitate PCR amplification by removal of the loop region, which will enable proper annealing of primers. Other information may also be encoded in the reaction site adapter hairpin DNA. One example is a series of varied bases incorporated in the loop region. When amplified after selection these varied bases will help identify library members that are being enriched in selection due to amplification biases or as artifacts. Another example is a specific sequence indicating information about the selection or synthesis history of the molecule that is like an index sequence as described in Example 7. Hairpins may also comprise fluorescently-labeled bases or base analogs, radiolabeled bases or base analogs, for quantitating and analyzing various aspects of the library and its synthesis or performance. Hairpins may also contain bases or modified bases bearing functional groups that facilitate processing, like biotin. Such hairpins can be purchased from reputable vendors of custom DNA oligos like IDT of Coralville, Iowa, Sigma Aldrich, or Eurofins MWG of Louisville, Ky.

### Example 51. Ligate a Reaction Site Adapter to a Template Strand with Other Chemistry

A reaction site adapter is annealed to the terminal coding region of a template gene as per Example 44 or 45. Other methods of covalently tethering the reaction site adapter can be used, including chemical or enzymatic methods. Some of such methods involve reactions using water soluble carbodiimide and cyanogen bromide as done by, Shabarova, et al. (1991) Nucleic Acids Research, 19, 4247-4251), Fed-erova, et al. (1996) Nucleosides and Nucleotides, 15, 1137-1147, GryaZnov, Sergei M. et al. J. Am. Chem. Soc., vol. 115:3808-3809 (1993), and Carriero and Darnlia (2003) Journal of Organic Chemistry, 68, 8328-8338. Chemical ligation is, in some cases, done using 5M cyanogen bromide in aceto-nitrile, in a 1:10 v/v ratio with 5′ phosphorylated DNA in a buffer containing 1M MES and 20 mM MgCl₂ at pH 7.6, the reaction being performed at 0 degrees for 5 minutes. Ligations can also be performed by topoisomerases, polymerases and ligases using manufacturer's protocols.

### Example 52. Prepare and Translate a Library with Single-Stranded Terminal Coding Regions

A library with less steric bulk is prepared by removing an oligonucleotide from the terminal coding region of the reaction site adapter to make the terminal coding region single stranded. In some cases, oligonucleotides are removed to make all or part of a stem region single-stranded. This is done exactly as per Example 44, 45, or 46, with the following exception. Deoxy-uridine is incorporated in the provided reaction site adapters at locations in the anti-coding sequence and the stem between the terminus of the anti-coding region and the nearest linker. After installation of the charged reaction site adapters to the template strand, the library is buffer exchanged into 1× UDG reaction buffer from NEB, uracil-DNA glycosylase (“UDG”) is added at a concentration of 20 U/ml and incubated 30 minutes at 37° C. as per the manufacturer's protocol. Subsequent heating to 95° C. at pH 12 for 20 minutes hydrolyzed the apyrimidinic sites in the hairpin. The small ssDNA fragments produced are removed by size exclusion executed with buffer kept at 65° C.

### Example 53. Removing an Oligonucleotide from the Terminal Coding Region of the Reaction Site Adapter is, in Some Cases, Performed at Several Points During the Execution of Example 44-46

The oligonucleotide can, in some cases, be removed exactly as per Example 52 with the exception that it is performed after ligation of the charged reaction site adapter, but before addition of a first positional building block. The oligonucleotide can, in some cases, be removed exactly as per Example 52 with the exception that the procedure is performed after addition of a first positional building block, but before addition of any subsequent positional building block. The oligonucleotide can, in some cases, be removed exactly as per Example 52 with the exception that the procedure is performed after addition of all positional building blocks. It will be appreciated by one skilled in the art that the task of cleaving a strand of DNA at a desired location is accomplished in many ways, and that there are a large number of commercially available enzymes and published protocols facilitating this task; for example, New England Bio Labs sells at least 10 nicking endonucleases and publishes protocols for their use. The specific examples given here are exemplary, and do not exclude other methods of accomplishing the task of making a terminal coding region and, in some cases, part of the hairpin single-stranded.

### Example 54a. Remove a 5′ Terminal Non-Coding Region Using UDG

The restriction digestion used to remove the 5′ terminal non-coding region in Example 44 and Example 45 is eliminated and replaced by treatment with UDG and subsequent alkaline hydrolysis of the apyrimidinic site. A library is prepared exactly as per Example 44, but wherein the oligo priming the reverse transcription incorporates a dU base at or near the 3′ end of the primer. After reverse transcription, and base hydrolysis of the RNA strand, UDG can remove uracil, creating an apyrimidinic site that is subsequently cleaved by heat and alkali (see example 5 for the use of UDG and reaction conditions) producing the terminal coding region that is ready for ligation of a charged reaction site adapter. It will be appreciated by one skilled in the art that there are a number of ways of cleaving a single strand or a double strand of DNA at a desired location, and that there are a large number of commercially available enzymes and published protocols facilitating this task. The specific examples given here are exemplary, and do not exclude other methods of accomplishing the task of removing a 5′ terminal non-coding region.

### Example 54b. Remove a 5′ Terminal Non-Coding Region or a 3′ Terminal Non-Coding Region Using the Restriction Enzyme NdeI

The restriction digestion used to remove the 5′ terminal coding region or the 3′ terminal coding region, or both, is accomplished by including the recognition site for NdeI in the terminal non-coding region, and performing restriction digestion after the reverse-transcription step. NdeI has the ability to cut RNA/DNA hybrids and also to cut single-stranded DNA. Thus, NdeI is used to cut either before or after base hydrolysis of the RNA strand, or both.

### Example 54c. Remove a 5′ Terminal Non-Coding Region Using RNA Bases in the Reverse Transcription Primer

The 5′ terminal non-coding region is removed using the exact protocol of Example 44-45, except the primer used in the step “reverse transcribe the RNA into DNA” contains an RNA base. Upon hydrolysis of the RNA strand of the reverse transcription product as per Example 3, the RNA base in the DNA primer will also hydrolyze, removing the portion of the DNA primer that is 5′ of the RNA base.

### Example 55. Prepare and Translate a Library with Alternative Reactive Site Functional Groups and Linkers

A library using a different initial reactive site from a free amine is made in several ways. One method is to cap an existing initial reactive site functional group with a bifunctional molecule bearing the desired initial reactive site functional group. Charge reaction site adapters exactly as per Example 44c, except that on each reaction site adapter on which a different initial reaction site is desired, a peptide bond is formed to the initial reactive site functional group amine with a bifunctional compound bearing a carboxylic acid and the desired initial reactive site functional group, using the peptide coupling reaction conditions listed in that step. For example, 5-hydroxy pentanoic acid could be reacted with the free amine to form a peptide bond, and establish the hydroxyl functional group as the initial reactive site for synthesizing the library.

A second method is to incorporate a different base modified with a different reactive site that enables or facilitates installation of other desired initial reactive site functional groups. One such base is 5-Ethynyl-dU-CE Phosphoramidite (“ethynyl-dU”) sold by Glen Research in Virginia. It is, in some cases, modified with a bifunctional linker compound bearing an azide and the desired initial reactive site functional group. For example, 5-azido pentanoic acid could be reacted with the alkynyl moiety in a “click” reaction (Huisgen reaction) with conditions found in Example 25, establishing the carboxylic acid as the initial reactive site functional group. As another representative but non-inclusive example, 5-azido 1-pentanal could be reacted with the alkynyl moiety in a “click” reaction (Huisgen reaction), establishing the aldehyde as the initial reactive site functional group. As another representative example, 4-azido, 1-bromomethylbenzene could be reacted with the alkynyl moiety in a “click” reaction (Huisgen reaction), establishing the benzyl halide as the initial reactive site functional group. In some embodiments, this base is used as an alkynyl initial reactive site for library synthesis using chemistries appropriate for alkynes chosen from Examples 9-31. Desirable initial reactive sites include, but are not limited to, amines, azides, carboxylic acids, aldehydes, alkenes, acryloyl groups, benzyl halides, halides alpha to carbonyl groups, and 1,3-dienes.

A third method is to incorporate a base modified with both a linker and an initial reactive site functional group during synthesis of the reaction site adapters. For example, incorporating 5′-Dimethoxytrityl-N6-benzoyl-N8-[6-(trifluoroacetylamino)-hex-1-yl]-8-amino-2′-deoxyAdenosine-3′-[(2-cyanoethyl)-(N,N-diisopropyl)]-phosphoramidite (also called amino-modifier C6 dA, purchased from Glen Research, Sterling Va.), at strategic locations during the synthesis of the adapter would establish a free amine as the initial reactive site functional group and a 6 carbon alkyl chain as the linker, as would incorporating 5′-Dimethoxytrityl-N2-[6-(trifluoroacetylamino)-hex-1-yl]-2′-deoxyGuanosine-3′-[(2-cyanoethyl)-(N,N-diisopropyl)]-phosphoramidite (also called amino-modifier C6 dG, purchased from Glen Research, Sterling, Va.). Incorporating 5′-Dimethoxytrityl-5-[3-methyl-acrylate]-2′-deoxyUridine,3′-[(2-cyanoethyl)-(N,N-diisopropyl)]-phosphoramidite (also called Carboxy dT, purchased from Glen Research, Sterling Va.) at strategic locations during the synthesis of the adapter would establish a carboxylic acid as the initial reactive site functional group and a 2 carbon chain as the linker. Incorporating 5′-Dimethoxytrityl-5-[N-((9-fluorenylmethoxycarbonyl)-aminohexyl)-3-acrylimido]-2′-deoxyUridine,3′-[(2-cyanoethyl)-(N,N-diisopropyl)]-phosphoramidite (also called Fmoc-amino modifier C6 dT, Glen Research, Sterling, Va.) at strategic locations during the synthesis of the adapter would establish an Fmoc-protected amine as the initial reactive site functional group and a 6 carbon alkyl chain as the linker. Incorporating 5′-Dimethoxytrityl-5-(octa-1,7-diynyl)-2′-deoxyuridine, 3′-[(2-cyanoethyl)-(N,N-diisopropyl)]-phosphoramidite (also called C8 alkyne dT, Glen Research, Sterling Va.) at strategic locations during the synthesis of the adapter would establish an alkyne as the initial reactive site functional group and an 8 carbon chain as the linker. Incorporating 5′-(4,4′-Dimethoxytrityl)-5-[N-(6-(3-benzoylthiopropanoyl)-aminohexyl)-3-acrylamido]-2′deoxyuridine, 3′-[(2-cyanoethyl)-(N,N-diisopropyl)]-phosphoramidite (also called S-B₂-Thiol-Modifier C6-dT, Glen Research, Sterling Va.) at strategic locations during the synthesis of the adapter would establish a thiol as the initial reactive site functional group and a 14 atom chain as the linker. Incorporating N4-TriGl-Amino 2′deoxycytidine (from IBA GmbH, Goettingen, Germany) at strategic locations during the synthesis of the adapter would establish an amine as the initial reactive site functional group and a 3-ethylene glycol unit chain as the linker.

Suitable linkers perform two critical functions: (i) they covalently tether the adapter (or template strand, or DNA coding strand, or library strand) to a building block, and (ii) they do not interfere with other critical functions in the synthesis or use of molecules of formula (I). Thus, in some embodiments, the linkers are alkyl chains or PEG chains because (a) they are highly flexible, allowing appropriate and free presentation of the encoded portions to target molecules during selections, and (b) because they are relatively chemically inert and typically do not undergo side reactions during synthesis of molecules of formula (I). To adequately perform most, but not all tasks, linkers need not comprise an overall length greater than about 8 PEG units. It will be appreciated by one skilled in the art that when performing selections in which the library DNA must be kept as far from the target molecule or target structure or target surface as possible, that considerably longer linkers, and/or considerably stiffer linkers, like a peptide alpha helix, would be useful and attractive. Other desirable linkers could include polyglycine, polyalanine, or polypeptides. Linkers are also used which incorporate a fluorophore, a radiolabel, or a functional moiety used to bind a molecule of formula (I) in a manner that is orthogonal to binding to the encoded portion, or that is complementary to the binding of the encoded portion. In some embodiments, a biotin is incorporated in the linker to immobilize the library. In other embodiments a known ligand is incorporated in one binding pocket of a target molecule to allow for performing selections for an encoded portion that can bind a second binding pocket of the same target molecule.

In some embodiments, libraries are prepared using different linkers and different chemistries on different reactive site adapters. The linker or linkers on the 5′ reaction site adapter can bear one type of linker, and one type of reactive site functional group, while the 3′ reaction site adapter bears a different linker and the same reactive site functional group, or a different linker and a different reactive site functional group. Any of the linkers and functional groups named herein are appropriate for use in this example provided the chemistries required for subsequent installation of positional building blocks is compatible with the functional groups on the first building block, D and second building block E, which are reacted with the reactive site functional groups on their respective hairpins.

This compatibility has two modes. In the first mode, different chemistries are used to charge the reaction site adapters, but both the first building block D and the second building block E are capable of undergoing the same chemical transformation in the next or a subsequent downstream step. In the second mode, different chemistries are used to charge the reaction site adapters, and different chemistries are required for a subsequent down stream step. This second mode requires that the functional groups on the nascent 5′ encoded portion, the functional groups on the incoming positional building block for the 5′ end, and the chemistry used for that coupling, is non-reactive with the functional groups present on the nascent 3′ encoded portion. Likewise, this second mode requires that the functional groups on the nascent 3′ encoded portion, the functional groups on the incoming positional building block for the 3′ end, and the chemistry used for that coupling, is non-reactive with the functional groups present on the nascent 5′ encoded portion. The steps of installing building blocks using orthogonal chemistries on the 3′ and 5′ reaction site adapters can be done in any order. In addition, it will be appreciated by one skilled in the art that among the diversity building blocks installed at a given step of synthesis, that not performing an installation of any building block is an important diversity element. Appropriate chemistries for these steps include but are not limited to the chemistries described in Examples 9-31 and Example 44-46.

### Example 56. Constructing a Gene Library with Corresponding Terminal Coding Regions

A library is constructed in which the 5′ terminal coding region and the 3′ terminal coding region encode the same building block or the same pair of different building blocks. This is accomplished if each member of the gene library possessing a given 5′ terminal coding sequence only possesses one 3′ terminal coding sequence. Such a library is constructed using the method of Example 2d except that the hybridized subset pairs for the 5′ terminal coding region and 3′ terminal coding region are not pooled. All internal coding regions are pooled and ligated as per example 2d. The product of ligating all the internal coding regions is split into aliquots and one aliquot is added to each 5′ terminal hybridized subset sequence and ligated. The ligation products in each well possess a single 5′ terminal coding sequence but a combinatorial mixture of all sequences at all internal coding regions. These ligation products with a single 5′ terminal coding sequence are transferred independently into wells containing a single 3′ terminal hybridized subset sequence and ligated. The product in each well is a gene comprising a single 5′ terminal coding sequence, a single 3′ terminal coding sequence, and a combinatorial mixture of all sequences at all internal coding regions. It will be appreciated that there are other ways of producing the same resultant library.

### Example 57. Coding Regions Comprised of Shorter DNA Sequences

Shorter sequences can be used for mononomial coding, multinomial coding, and non-coding regions. Incorporating certain modified bases into the capture oligos, or into reaction site adapter coding sequences will increase the Tm of the hybrid formed between such a capture oligo and the coding strand, making shorter coding sequences as efficient as longer sequences. Modified bases that can be used to accomplish this task include but are not limited to: 2-amino-dA, 5-methyl dC, 5-propynyl dC, 7-propynyl-8-aza-7-deazapurin-2,6-diamine 2′deoxyribonucleosides, and Locked Nucleic Acids (LNAs). (See refs. (a) Y. Lebedev, et al., Genetic Analysis—Biomolecular Engineering, 1996, 13, 15-21. (b) L. E. Xodo, G. Manzini, F. Quadrifoglio, G. A. v. d. Marel, and J. H. v. Boom, Nucleic Acids Res., 1991, 19, 5625-5631. (c) B. C. Froehler, S. Wadwani, T. J. Terhorst, and S. R. Gerrard, Tetrahedron

1-8. (canceled)

9. A molecule according to formula (I),
[(B₁)_{M}-L₁]_{O}-G-[(L₂-(B₂)_{K}]_{P}
wherein
G includes an oligonucleotide, the oligonucleotide comprising at least two coding regions, wherein the at least two coding regions are single stranded;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G;
L₂ is a linker that operatively links B₂ to G;
O is zero or 1;
P is zero or 1;
provided that at least one of O and P is 1; and
wherein each positional building block B₁ at position M and/or B₂ at position K is identified by from 1 to 5 coding regions, and from about 10% to 100% of the positional building blocks B₁ at position M and/or B₂ at position K, based on a total number of positional building blocks, are identified by a combination of from 2 to 5 independent coding regions.

10. The molecule of claim 9, wherein G comprises a sequence represented by the formula (C_{N}-(Z_{N}-C_{N+1})_{A}) or (Z_{N}-(C_{N}-Z_{N+1})_{A}), wherein C is a coding region, Z is a non-coding region, N is an integer from 1 to 20, and A is an integer from 1 to 20; wherein each non-coding region contains from 0 to 50 nucleotides and is optionally double stranded.

11. The molecule of claim 9, wherein each coding region contains from 6 to 50 nucleotides, or wherein each coding region contains from 8 to 30 nucleotides.

12. (canceled)

13. The molecule of claim 9, wherein at least one of O or P is zero.

14. The molecule of claim 9, wherein from about 20% to 100% of the positional building blocks B₁ at position M and/or B₂ at position K, based on the total number of positional building blocks, are identified by a combination of from 2 to 5 independent coding regions, or
wherein from about 20% to 100% of the positional building blocks B₁ at position M and/or B₂ at position K, based on the total number of positional building blocks, are identified by a combination of from 2 to 3 independent coding regions.

15. (canceled)

16. The molecule of claim 9, wherein P is 0; O is 1; and
from about 30% to 100% of the positional building blocks B₁ at position M, based on the total number of positional building blocks, are identified by a combination of from 2 to 3 independent coding regions; or
wherein O is 0; P is 1; and from about 30% to 100% of the positional building blocks B₂ at position K, based on the total number of positional building blocks, are identified by a combination of from 2 to 3 independent coding regions.

17. (canceled)

18. A method of identifying probe molecules capable of binding or selecting for a target molecule comprising:
exposing the target molecule to a pool of probe molecules, wherein the probe molecules are according to claim 1,
removing at least one probe molecule that does not bind the target molecule,
amplifying the oligonucleotide of G from the at least one probe molecule that was not removed from the target molecule to form a copy sequence,
sequencing the copy sequence to identify each coding region and combination of coding regions of the probe molecule to further identify each positional building block B₁ at position M and/or B₂ at position K.

19. The method of claim 18, comprising:
sequencing the copy sequence to identify each coding region and combination of from 2 to 3 independent coding regions of the probe molecule to further identify at least one of each positional building block B₁ at position M and B₂ at position K.

20. A method of forming a molecule of formula (I) comprising: providing at least one first hybridization array, the at least one first hybridization array comprising at least one first single stranded anti-codon oligomer immobilized on the at least one first hybridization array, wherein the at least one first single stranded anti-codon oligomer immobilized on the at least one first hybridization array is capable of hybridizing to a first coding region of a molecule of formula (II):
[(B₁)_{(M-1)}-L₁]_{O}-G-[(L₂-(B₂)_{(K-1)})_{P} (II)
wherein
G includes an oligonucleotide, the oligonucleotide comprising at least two coding regions, wherein the at least two coding regions are single stranded;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G;
L₂ is a linker that operatively links B₂ to G;
O is zero or 1;
P is zero or 1;
provided that at least one of O and P is 1; and
wherein each positional building block B₁ at position M and/or B₂ at position K is identified by from 1 to 5 coding regions, and from about 10% to 100% of the positional building blocks B₁ at position M and/or B₂ at position K, based on a total number of positional building blocks, are identified by a combination of from 2 to 5 independent coding regions;
sorting the pool of molecules of formula (II) into a first set of sub-pools by hybridizing the first coding region of the molecules of formula (II) to the at least one first single stranded anti-codon oligomer immobilized on the at least one first hybridization array;
releasing the first set of sub-pools of molecules of formula (II) from the at least one first hybridization array into separate containers;
providing at least one second hybridization array, the at least one second hybridization array comprising at least one second single stranded anti-codon oligomer immobilized on the at least one second hybridization array, wherein the at least one second single stranded anti-codon oligomer immobilized on the at least one second hybridization array is capable of hybridizing to a second coding region of a molecule of formula (II):
independently sorting each, or at least one, of the first set of sub-pools of molecules of formula (II) into a second set of sub-pools by hybridizing the second coding region of the molecules of formula (II) to the at least one second single-stranded anti-codon oligomer immobilized on the at least one second hybridization array;
providing at least one of building block B₁ and B₂; and
reacting the at least one of building block B₁ and B₂ with the molecule of formula (II) to form a sub-pool of molecules of formula (I):
[(B₁)_{M}-L₁]_{O}-G-[(L₂-(B₂)_{K})_{P}
wherein
G includes an oligonucleotide, the oligonucleotide comprising at least two coding regions,
wherein the at least two coding regions are single stranded;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G;
L₂ is a linker that operatively links B₂ to G;
O is zero or 1;
P is zero or 1;
provided that at least one of O and P is 1; and
wherein each positional building block B₁ at position M and/or B₂ at position K is identified by from 1 to 5 coding regions, and from about 10% to 100% of the positional building blocks B₁ at position M and/or B₂ at position K, based on a total number of positional building blocks, are identified by a combination of from 2 to 5 independent coding regions.

21. The method of claim 20, further comprising, before the step of the reaction step,
releasing the second set of sub-pool of molecules of formula (II) from the at least one second hybridization array into a second set of separate containers;
providing at least one third hybridization array, the at least one third hybridization array comprising at least one third single stranded anti-codon oligomer immobilized on the at least one third hybridization array, wherein the at least one third single stranded anti-codon oligomer immobilized on the at least one third hybridization array is capable of hybridizing to a third coding region of a molecule of formula (II);
independently sorting at least one sub-pool from the second set of sub-pools of molecules of formula (II) into a third set of sub-pools by hybridizing the third coding region of the third set of sub-pools of molecules of formula (II) to the at least one third single stranded anti-codon oligomer immobilized on the at least one third second hybridization array; and
optionally, repeating steps (a), (b), and (c).

22. The method of claim 20, wherein each coding region contains from 6 to 50 nucleotides, or
wherein each coding region contains from 8 to 30 nucleotides.

23. (canceled)

24. The method of claim 20, wherein at least one of O or P is zero.

25. The method of claim 20, wherein from about 20% to 100% of the positional building blocks B₁ at position M and/or B₂ at position K, based on a total number of positional building blocks, are identified by a combination of from 2 to 5 independent coding regions, or
wherein from about 20% to 100% of the positional building blocks B₁ at position M and/or B₂ at position K, based on a total number of positional building blocks, are identified by a combination of from 2 to 3 independent coding regions.

26. (canceled)

27. The method of claim 21, wherein P is 0; O is 1; and from about 30% to 100% of the positional building blocks B₁ at position M, based on the total number of positional building blocks, are identified by a combination of from 2 to 3 independent coding region; or
wherein O is 0; P is 1; and from about 30% to 100% of the positional building blocks B₂ at position K, based on the total number of positional building blocks, are identified by a combination of from 2 to 3 independent coding regions.

28. (canceled)

29. A method of forming an oligonucleotide-encoded molecule comprising:
providing at least one first hybridization array, the at least one first hybridization array comprising at least one first single stranded anti-codon oligomer immobilized on the at least one first hybridization array, wherein the at least one first single stranded anti-codon oligomer immobilized on the at least one first hybridization array is capable of hybridizing to a first coding region of an oligonucleotide molecule G comprising:
at least a first coding region and a second coding region, wherein the first and second coding regions are single-stranded and wherein the first and second coding regions are different; and
a reactive site either on the 3′ terminus of G, or an internal nucleotide 5′ to the at least a first and second coding region, or an internal nucleotide 3′ to the at least a first and second coding region;
(b) sorting a pool of oligonucleotides G into a first set of sub-pools by hybridizing the first coding region of the oligonucleotide to the at least one first single stranded anti-codon oligomer immobilized on the at least one first hybridization array;
(c) providing at least one second hybridization array, the at least one second hybridization array comprising at least one second single stranded anti-codon oligomer immobilized on the at least one second hybridization array, wherein the at least one second single stranded anti-codon oligomer immobilized on the at least one second hybridization array is capable of hybridizing to the second coding region of the oligonucleotide;
(d) independently sorting at least one of the first set of sub-pools of the oligonucleotide into a second set of sub-pools by hybridizing the second coding region of the oligonucleotide to the at least one second single-stranded anti-codon oligomer immobilized on the at least one second hybridization array.

30-60. (canceled)
